(19) United States
(12) Patent Application Publication
(10) Pub. No.: US 2026/0097072 A1
(43) Pub. Date: Apr. 9, 2026

(54) FORMULATIONS OF AMMONIUM CHLORIDE TO SUPPORT HUMAN NATURAL DEFENSE AGAINST VIRUSES

(71) Applicant: Nikolaos Tsirikos-Karapanos, Cleveland, OH (US)

(72) Inventors: Nikolaos Tsirikos-Karapanos, Cleveland, OH (US); Nikolaos Drakoulis, Athens (GR)

(21) Appl. No.: 19/349,399

(22) Filed: Oct. 3, 2025

Related U.S. Application Data
(60) Provisional application No. 63/702,795, filed on Oct. 3, 2024.

(51) Int. Cl. A61K 33/20; A61K 9/20; A61K 9/28; A61K 9/48; A61K 31/593; A61P 31/14; A61P 31/16

(52) U.S. Cl. CPC A61K 33/20; A61K 9/2013; A61K 9/2027; A61K 9/28; A61K 9/4866; A61K 31/593; A61P 31/14; A61P 31/16

(57) ABSTRACT

Formulations and uses thereof are provided for supporting human natural defense against viral infections as well as providing treatment for viral infections susceptible to a lysosomotropic agent. Administration of a lysosomotropic agent, such as ammonium chloride (NH₄Cl), can militate against the uncoating of viruses within the lysosome of an infected cell and thereby minimize infection by viruses whose replication cycle relies upon an uncoating step in such a manner.

### CROSS-REFERENCE TO RELATED APPLICATIONS

This application claims the benefit of U.S. Provisional Application No. 63/702,795, filed on Oct. 3, 2024. The entire disclosure of the above application is incorporated herein by reference.

### FIELD

The present technology relates to novel dietary supplement formulations and pharmaceutical formulations of a lysosomotropic agent, where dietary supplement formulations of the lysosomotropic agent can be used to support the natural defense of a subject against viral infections, and where pharmaceutical formulations of the lysosomotropic agent can be used to provide treatment for viral infections.

### BACKGROUND

This section provides background information related to the present disclosure, which is not necessarily prior art.

Various viruses are responsible for serious diseases in various subjects, including animals and humans, including the Influenza A and Influenza B viruses, the Respiratory Syncytial Virus (SRV), the Syncytial Acute Respiratory Syndrome Coronavirus (SARS-CoV), the SARS-CoV-2 and the Middle East Respiratory Syndrome Virus (MERS). Certain viruses can infect subjects of different species, whereas other viruses can be host species specific.

The first identified strain of SARS-CoV was identified in 2003 in China. SARS-CoV is believed to be an animal virus from an as-yet-uncertain animal reservoir (e.g., bats) that spread to other animal species (e.g., cats) and then infected humans; first, in southern China in 2002. SARS-CoV is primarily transmitted from person-to-person due to virus excretion in respiratory secretions. Twenty-six countries were affected during the SARS epidemic with more than 8,000 cases reported in 2003.

SARS-CoV-2 is the pathogen leading to coronavirus disease (COVID-19). The 2020 COVID-19 pandemic is having a serious global socioeconomic impact. As of November 2020, more than 11,000,000 COVID-19 cases have been confirmed in the U.S. and more than 250,000 people have died from COVID-19 disease. COVID-19 symptoms may appear 2-14 days after the exposure to the SARS-CoV-2 virus. The clinical manifestations of the COVID-19 disease vary. In general, fever, cough, and shortness of breath are common clinical manifestations of the COVID-19 disease accompanied by chills, ageusia (loss of taste), anosmia (loss of smell), muscular aches, headache, sore throat, and gastrointestinal symptoms (vomiting and diarrhea). COVID-19 patients symptoms range from being asymptomatic or having minor respiratory symptoms up to having serious respiratory and general symptoms requiring hospitalization and/or ICU treatment, including endotracheal intubation and respiratory support by a mechanical ventilator. Symptomatic and asymptomatic COVID-19 patients can have similar viral loads indicating that patients can transmit the virus regardless of the severity of their symptoms. This makes it very difficult to control of the spread of the disease.

There are limited etiological therapies for the COVID-19 disease available, including certain vaccines and antiviral agents, where treatment of COVID-19 patients is sometimes limited to alleviation of symptoms and provision of organ support as needed by the individual patient. Not all patients have access to certain therapies and not all therapies available to date are applicable to certain patients. Accordingly, there is a continuing need for ways to treat COVID-19 patients.

### SUMMARY

In concordance with the instant disclosure, the present technology includes articles of manufacture, systems, and processes that relate to supporting human natural defense against viral infections as well as providing treatment for viral infections susceptible to a lysosomotropic agent. Various ways are provided to support natural defenses against viral infections and/or provide treatment of viral infections in a subject, such as a human or animal subject. Administration of a lysosomotropic agent, such as ammonium chloride (NH₄Cl), can be used to minimize clinical manifestations of viral infections, including infection by the SARS-CoV-2 virus, the Influenza-A virus, and the Influenza-B virus.

Certain embodiments include methods of militating against a clinical manifestation of infection by a virus in a subject. Such methods can administer a formulation to the subject, where the formulation includes a lysosomotropic agent, the lysosomotropic agent thereby interfering with viral uncoating, providing a viral-static effect, and allowing immunological mechanisms of the subject to produce antibodies against the virus. The lysosomotropic agent can include ammonium chloride and the formulation can be configured in a unit dosage form. The formulation can include an enteric coating configured for oral administration. The lysosomotropic agent can be comprised by microspheres that provide a sustained release of the lysosomotropic agent, where the microspheres are enclosed by the enteric coating.

Certain embodiments include formulations for militating against a clinical manifestation of infection by a virus in a subject. Such formulations can include a lysosomotropic agent, such as ammonium chloride in an amount from about 10 mg to about 2,000 mg. The formulation can be configured in a unit dosage form and can include an enteric coating configured for oral administration. Various excipients can be included in the formulation, such as sodium croscarmellose in an amount from about 10 mg to about 200 mg, hydroxypropyl methylcellulose in an amount from about 10 mg to about 200 mg, and/or magnesium stearate in an amount from about 0.1 mg to about 40 mg. The lysosomotropic agent can be comprised by microspheres that provide a sustained release of the lysosomotropic agent following oral administration, where the microspheres are enclosed by the enteric coating. The formulation can also include vitamin D in an amount from about 1,000 to about 4,000 IU.

The lysosomotropic agent can be used to militate against clinical manifestation of a viral infection in a subject, including infection by the SARS-CoV-2 virus, the Influenza-A virus, and the Influenza-B virus. The lysosomotropic agent can prevent viral uncoating in the subject, thereby resulting in a viral-static effect that can be adequate to provide the necessary time for mounting of natural defense mechanisms in the subject. In particular, the viral-static effect can permit the immune system of the subject enough time to produce a sufficient number of antibodies against the virus to mount an effective immunological response thereto.

Certain formulations of the present technology can include: vitamin D (1,000-4,000 IU); NH₄Cl (250-500 mg); acetyl alcohol (50-400 mg); stearyl alcohol (50-400 mg); polyvinylpyrrolidone (1-20 mg); magnesium stearate (2-20 mg); ethanol (0-100 mg); purified water (10-250 mg); and a coating system (0-30 mg).

In certain embodiments, a formulation for militating against a clinical manifestation of infection by a virus in a subject is provided. The formulation can include a lysosomotropic agent including ammonium chloride in an amount from about 10 mg to about 2,000 mg. The formulation can be configured in a unit dosage form and can include an enteric coating configured for oral administration that provides a sustained release of the ammonium chloride. The formulation can have a dissolution profile from 4 hours to 24 hours of full release of ammonium chloride. The dissolution profile can be determined by U.S. Pharmacopeial Convention Monograph USP <711>. The formulation can include a secosteroid and the secosteroid can include vitamin D, for example, as vitamin D₂ and/or vitamin D₃. Certain embodiments of the formulation can include the ammonium chloride at 250-1,500 mg, vitamin D, and an excipient including a member selected from a group consisting of steric acid, acetyl alcohol, stearyl alcohol, polyvinylpyrrolidone, magnesium stearate, and combinations thereof. The formulation can include embodiments having the ammonium chloride at 250-1,500 mg, the vitamin D at 1,000-4,000 IU, and where the excipient includes steric acid, acetyl alcohol, stearyl alcohol, polyvinylpyrrolidone, and magnesium stearate.

In certain embodiments, formulations of the present technology can include the following aspects. The lysosomotropic agent can be in an amount effective to reduce intracellular viral load at 24 hours post-infection. The lysosomotropic agent can be in an amount effective to reduce intracellular viral load from 24 hours post-infection up to 72 hours post-infection. The lysosomotropic agent can be in an amount effective to increase a threshold cycle for detecting intracellular viral load by RT-PCR at 72 hours post-infection by at least 25% for SARS-CoV-2. The lysosomotropic agent can be in an amount effective to increase a threshold cycle for detecting intracellular viral load by RT-PCR at 72 hours post-infection by at least 100% for influenza-A.

In certain embodiments, a formulation of the present technology can have a dissolution profile from 4 hours to 24 hours of full release of ammonium chloride where the dissolution profile is determined by U.S. Pharmacopeial Convention Monograph USP <711>. The formulation can have the ammonium chloride at 250-1,500 mg, vitamin D, and an excipient including a member selected from a group consisting of steric acid, acetyl alcohol, stearyl alcohol, polyvinylpyrrolidone, magnesium stearate, and combinations thereof. The lysosomotropic agent can be in an amount effective to reduce intracellular viral load at 24 hours post-infection.

In certain embodiments, a method of militating against a clinical manifestation of infection by a virus in a subject is provided. The method can include administering a formulation to the subject, the formulation including a lysosomotropic agent having ammonium chloride in an amount from about 10 mg to about 2,000 mg, the formulation configured in a unit dosage form, and the formulation having an enteric coating configured for oral administration that provides a sustained release of the ammonium chloride. The method can include monitoring the subject for at least one of alkalosis and acidosis, wherein the monitoring includes measuring a blood pH of the subject. The method can include selectively adjusting the administering of the formulation to the subject based on the monitoring so that the subject is not exhibiting alkalosis and is not exhibiting acidosis, wherein the selectively adjusting includes adjusting the administering of the formulation to the subject until the blood pH of the subject is between about 7.35 and about 7.45. And the method can include continuing the administering of the formulation over a period of at least 24 hours to reduce intracellular viral load at 24 hours post-infection.

Methods of the present technology can further include the following aspects. For example, continuing the administering of the formulation can include continuing administration of the formulation over a period of 72 hours to reduce intracellular viral load at 72 hours post-infection. The formulation can have a dissolution profile from 4 hours to 12 hours of full release of ammonium chloride and the formulation can include the ammonium chloride at 250-500 mg, vitamin D, and an excipient including a member selected from a group consisting of steric acid, acetyl alcohol, stearyl alcohol, polyvinylpyrrolidone, magnesium stearate, and combinations thereof. The virus can include SARS-CoV-2 virus, the Influenza-A virus, or the Influenza-B virus, and the lysosomotropic agent can be in an amount effective to increase a threshold cycle for detecting intracellular viral load by RT-PCR at 72 hours post-infection by at least 25% for SARS-CoV-2. The virus can include influenza-A virus and the influenza-B virus and the lysosomotropic agent can be in an amount effective to increase a threshold cycle for detecting intracellular viral load by RT-PCR at 72 hours post-infection by at least 100% for influenza-A. The method can include continuing the administration of the formulation to the subject until the virus cannot be detected in the subject. The method can also include continuing the administration of the formulation to the subject until an antibody to the virus is detected in the subject.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes and are not intended to limit the scope of the present disclosure.

FIG. 1 depicts schematic representations of various SARS-CoV-2 virus and host cell (e.g., animal or human cell) components referenced in the present disclosure, identified as follows: (A) SARS-CoV-2 virus, (B) SARS-CoV-2 surface spike protein (“S protein”), (C) SARS-CoV-2 genetic material (RNA), host cell (human cell), (E) Angiotensin Converting Enzyme II (“ACE-2”) receptor, (F) lysosome of the host cell.

FIG. 2 depicts the natural course of the SARS-CoV-2 virus infection of a host cell, with reference to the schematic representations of the components identified in FIG. 1.

FIG. 3 graphically depicts physiologic or acceptable blood pH variation overtime.

FIG. 4 depicts schematic representations of components used in the preparation of an ammonium chloride formulation according to an embodiment of the present technology, where the lysosomotropic agent NH₄Cl and excipients are mixed and formulated in microspheres that are further inserted into an enteric coated softgel or into an enteric coated capsule capable of sustained release of the lysosomotropic agent after the enteric coated softgel or the enteric coated capsule dissolves at the small bowel.

FIG. 5 depicts schematic representations of components used in the preparation of an ammonium chloride formulation according to an embodiment of the present technology, where the lysosomotropic agent NH₄Cl and the excipients are mixed and formulated in a sustained release enteric coated softgel or enteric coated capsule formulation capable of sustained release of the lysosomotropic agent after the enteric coated softgel or the enteric coated capsule dissolves at the small bowel.

FIG. 6 depicts a schematic representation of the blood concentration of NH₄Cl after its release from the sustained release formulation and its absorption, where Cmin represents a minimum effective NH₄Cl blood concentration, Cmax represents a minimum toxic NH₄Cl blood concentration, and [Cmax−Cmin] represents an effective NH₄Cl blood concentration window.

FIG. 7 depicts a schematic representation of a basket apparatus used to determine a dissolution profile of a formulation constructed in accordance with the present technology.

FIG. 8 graphically depicts experimentation results with respect to SARS-CoV-2 intracellular viral load in SARS-CoV-2 infected cell culture following an effective NH₄Cl dose.

FIG. 9 graphically depicts experimentation results with respect to SARS-CoV-2 extracellular viral load in SARS-CoV-2 infected cell culture following an effective NH₄Cl dose.

FIG. 10 graphically depicts experimentation results with respect to Influenza-A intracellular viral load in Influenza-A infected cell culture following an effective NH₄Cl dose.

FIG. 11 graphically depicts experimentation results with respect to Influenza-A extracellular viral load in Influenza-A infected cell culture following an effective NH₄Cl dose.

FIG. 12 graphically depicts longitudinal Ct values trajectories per group for an example embodiment of the present technology, where Ct: cycle threshold; SD: standard deviation; ACF: ammonium chloride formulation; VDF: vitamin D formulation; RT-PCR: reverse-transcriptase polymerase chain reaction.

FIG. 13 graphically depicts clearance by Day 10-11 (Ct≥40): ACF vs VDF with 95% CI for an example embodiment of the present technology, where Ct: cycle threshold; ACF: ammonium chloride formulation; VDF: vitamin D formulation; vs: versus; 95% CI: confidence interval.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The present technology includes ways of militating against a clinical manifestation of infection by a virus in a subject, including infection by the SARS-CoV-2 virus. Methods are provided that include administering a formulation to the subject, where the formulation includes a lysosomotropic agent. The lysosomotropic agent can therefore interfere with viral uncoating, provide a viral-static effect, and allow immunological mechanisms of the subject to produce antibodies against the virus. The present technology includes aspects described in U.S. patent application Ser. No. 17/532,582 to Tsirikos-Karapanos et al., filed Nov. 22, 2021, which is incorporated herein by reference.

The administered formulation can include various aspects. The lysosomotropic agent can include ammonium chloride and the formulation can be configured in a unit dosage form. Certain embodiments include where the formulation has an enteric coating configured for oral administration. The enteric coating can be configured to release formulation components in the gastrointestinal tract after the stomach, for example, in the duodenum or upper tract of the intestine. The lysosomotropic agent can be present in or take the form of microspheres that provide a sustained release of the lysosomotropic agent. The microspheres themselves can be enclosed by the enteric coating. For example, the enteric coating can take the form of a softgel or capsule that can enclose a quantity of microspheres. The formulation can include one or more excipients, such as sodium croscarmellose, hydroxypropyl methylcellulose, and/or magnesium stearate. Embodiments of the formulation can further include vitamin D.

The methods of militating against a clinical manifestation of infection by a virus in a subject can further include various aspects. Embodiments include adjusting the administering of the formulation to the subject so that the subject is not exhibiting alkalosis and not exhibiting acidosis. For example, certain methods can include measuring a blood pH of the subject and adjusting the administering of the formulation to the subject until the blood pH of the subject is between about 7.35 and about 7.45. It is further possible to continue the administration of the formulation to the subject until an antibody to the virus is detected in the subject. It is also further possible to continue the administration of the formulation to the subject until the virus cannot be detected in the subject; for example, where the subject exhibits a negative viral test. The negative viral test can be based upon a polymerase chain reaction (PCR) test or reverse-transcription polymerase chain reaction (RT-PCR) test for viral genetic material and/or an antibody-based test for a viral antigen.

Certain embodiments of the present technology can utilize formulations with the following aspects. The lysosomotropic agent can include ammonium chloride in an amount from about 10 mg to about 2,000 mg per day. The formulation can further includes: sodium croscarmellose in an amount from about 10 mg to about 200 mg a day; hydroxypropyl methylcellulose in an amount from about 10 mg to about 200 mg a day; and/or magnesium stearate in an amount from about 0.1 mg to about 40 mg a day. The formulation can be configured in a unit dosage form and can include an enteric coating configured for oral administration. The ammonium chloride, the sodium croscarmellose, the hydroxypropyl methylcellulose, and the magnesium stearate can be comprised by microspheres, where the microspheres are enclosed by the enteric coating and provide a sustained release of the ammonium chloride.

Certain embodiments of the present technology include formulations configured for external topical use and administering the formulation to the subject can include application of the formulation to subject's skin. The formulation can be configured for sustained release of the lysosomotropic agent to the subject's skin.

The present technology further provides formulations for militating against a clinical manifestation of infection by a virus in a subject. Such formulations can have a lysosomotropic agent including ammonium chloride in an amount from about 10 mg to about 2,000 mg. The formulation can be configured in a unit dosage form, such as a solid unit dosage form, and can include an enteric coating configured for oral administration. As described for uses of the formulation, the formulation can further include various excipients, such as sodium croscarmellose in an amount from about 10 mg to about 200 mg per day, hydroxypropyl methylcellulose in an amount from about 10 mg to about 200 mg per day, and/or magnesium stearate in an amount from about 0.1 mg to about 40 mg per day. The lysosomotropic agent can be enclosed by an enteric coating that is configured for sustained release of the lysosomotropic agent. The lysosomotropic agent can be comprised by microspheres to provide a sustained release of the lysosomotropic agent, where an amount of microspheres can be enclosed by the enteric coating. Certain formulations can further include vitamin D in an amount from about 1,000 to about 9,000 IU per day.

The present technology takes advantage of aspects of the replication cycle of certain viruses, including SARS-CoV-2. Viruses cannot replicate themselves; they need to insert into a host cell in order to replicate. Once a virus enters into the host cell, the virus needs to release its genetic material (e.g., RNA for SARS-CoV-2) into the host cell cytoplasm and use the host cell “machinery” to produce the elements needed for the replication of the virus. This results in the intracellular production of several viruses that, after the host cell's death, are released and are ready to infect other healthy host cells.

With respect to SARS-CoV-2, particular features of the virus operate at certain points in the replication cycle. The SARS-CoV-2 virus surface spike protein (“S protein”) has a high affinity for the ACE-2 receptor of various host cells (e.g., human host cells). Human cells with ACE-2 receptors on their surface, for example, include Type-II alveolar cells in the lung, as well as cells in several extra-pulmonary tissues, including cells in the heart, kidneys, and intestines. The genetic material of the SARS-CoV-2 virus is RNA. For the SARS-CoV-2 virus to replicate, it needs to release its RNA into the cytoplasm of a host cell.

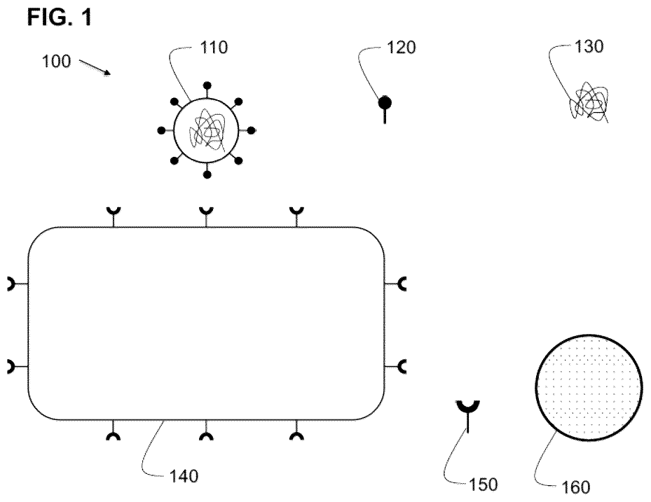

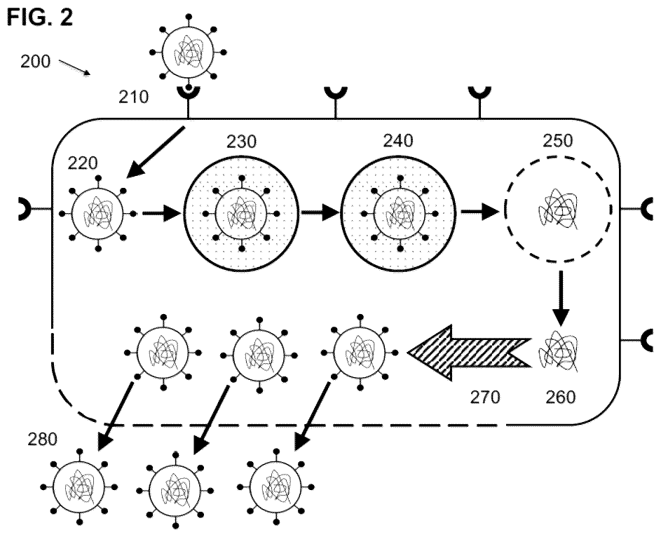

FIG. 1 shows schematic representations of components 100 of the SARS-CoV-2 virus and the host cell (e.g., animal or human cell), including the following: SARS-CoV-2 virus 110, SARS-CoV-2 surface spike protein (“S protein”) 120, SARS-CoV-2 genetic material (RNA) 130, host cell (human cell) 140, Angiotensin Converting Enzyme II (“ACE-2”) receptor 150, lysosome of the host cell 160. The general representations of these components are used in the viral infection steps shown in FIG. 2. It should be understood that the components depicted in FIGS. 1-2 and the steps shown in FIG. 2 are for general reference of certain aspects and events in viral infection and replication, and are not intended to be complete or rigidly define such. Other viral and host components may be involved and stages of viral infection and replication are not necessarily discrete or limited to the depicted features.

The natural course of the SARS-CoV-2 virus infection of a host cell and SARS-CoV-2 virus replication generally includes the following aspects. Once the SARS-CoV-2 virus infects a human, for example by entering into the human respiratory system, the following general sequence of steps occurs, as illustrated by the steps shown at 200 in FIG. 2. At step 210, the SARS-CoV-2 virus S protein “anchors” at the ACE-2 receptor of the host cell. At step 220, the SARS-CoV-2 virus enters into the host cell cytoplasm via a receptor-mediated endocytosis mechanism. At step 230, once inside the host cell cytoplasm, the SARS-CoV-2 virus enters into a host cell's lysosome. The normal lysosome pH is acidic ranging from around 4.5 to 5.0. At step 240, inside the host cell's lysosome and under the influence of lysosomal degenerative enzymes (e.g., lysosomal hydrolases) the SARS-CoV-2 viral membrane breaks. This process, known as “uncoating,” is pH dependent. Lysosomal hydrolases causing the SARS-CoV-2 virus uncoating include enzymes active only at the acidic lysosomal pH (4.5-5.0), which become inactive at less acidic pH and in neutral pH. In order to maintain an acidic lysosomal state within the range of pH 4.5 to 5.0, lysosomal membrane proteins pump protons (i.e., H⁺ ions) from the cytoplasm of the cell into the lysosome. The cytoplasm pH is slightly alkaline (e.g., about pH 7.2). At step 250, the SARS-CoV-2 virus uncoating results in the SARS-CoV-2 RNA being released into the lysosome. At step 260, the SARS-CoV-2 RNA is released into the cytoplasm. At step 270, once in the cytoplasm, the SARS-CoV-2 RNA directs the host cell machinery to start translating the SARS-CoV-2 RNA into viral proteins, including an RNA dependent RNA polymerase that replicates the viral RNA, resulting in SARS-CoV-2 viral replication. At step 280, completion of the intracellular SARS-CoV-2 viral replication is followed by host cell death and release of newly assembled SARS-CoV-2 viruses that are now ready to infect other host cells. Post human infection, the process of SARS-CoV-2 replication can be very fast, and the depicted steps are repeated in high speed, where the virus can infect and subsequently kill a very large number of host cells.

There are various aspects of host defense that relate to viral infection. By the moment that the SARS-CoV-2 virus enters the human body, the natural human defense mechanism of antibody formation against the SARS-CoV-2 virus is triggered and specific anti-SARS-CoV-2 virus antibodies begin to be produced. Unfortunately, this natural defense mechanism may be inadequate to protect the host, as the process to produce specific anti-SARS-CoV-2 antibodies in an amount effective against the virus may be significantly slower than the SARS-CoV-2 virus replication rate.

The present disclosure provides a lysosomotropic agent, such as ammonium chloride (NH₄Cl), in certain formulations, such as novel dietary supplements and pharmaceutical formulations, that cause a reversible increase of lysosomal pH in the cells of a subject and thus result in blocking the uncoating step of the SARS-CoV-2 virus. Failure to uncoat the SARS-CoV-2 virus, or even pausing or interruption of the uncoating process, as described above and shown in FIG. 2, due to an increased lysosomal pH (i.e., less acidic) can have a viral-static effect. The viral-static effect can significantly delay the SARS-CoV-2 viral replication rate and allow sufficient time for adequate anti-SARS-CoV-2 antibodies to be produced by the infected subject to effectively fight against the SARS-CoV-2 virus infection. The viral-static effect of the lysosomotropic agent alone or in combination with other therapeutic approaches can significantly minimize the clinical manifestations of the COVID-19 disease and reduce the need for hospitalization, ICU treatment, and/or the need for endotracheal intubation and mechanical ventilation. Various formulations including the lysosomotropic agent and various treatment modalities are provided by the present technology.

In certain embodiments, the lysosomotropic agent includes ammonium chloride (NH₄Cl), which is a water-soluble salt. Ammonium chloride is a lysosomotropic agent that can act at the lysosome to reversibly increase the lysosomal pH by blocking the lysosome membrane proteins pumping protons (H⁺ ions) from the cytoplasm into the lysosome. Christian De Duve introduced the term “lysosomotropism” in 1974 to describe substances that act selectively at the lysosome (“lysosomotropic” substances). From a regulatory point of view, ammonium chloride is designated as having a Generally Recognized As Safe status (GRAS status) from the U.S. Food & Drug Administration (see 21 CFR § 184.1138) and can be lawfully used in various formulations, including dietary supplements and in pharmaceutical formulations.

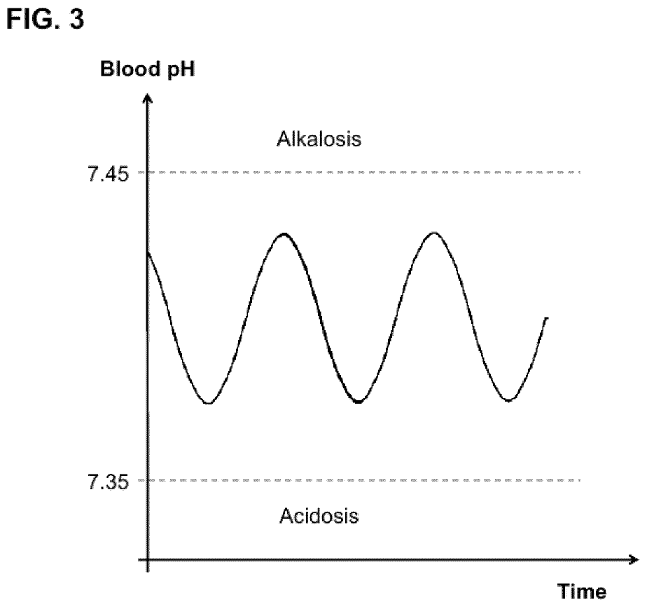

Ammonium chloride can further function as excipient in various formulations, including cough and cold medications, and can act as an expectorant to help clear lung secretions. Intravenous (“IV”) administration of ammonium chloride can be used for electrolyte replenishment (mainly hypochloremia) and in the treatment of alkalosis. Ammonium chloride is contraindicated in patients with severe impairment of renal or liver function. Rapid IV ammonium chloride administration or ammonium chloride overdose can result in serious metabolic acidosis, disorientation, confusion, and coma. If administered by mouth, ammonium chloride can cause upset stomach.

Where the lysosomotropic agent includes ammonium chloride, the ammonium chloride can alter the pH at various portions of the subject to which it is administered. The blood of the subject, in particular, is typically physiologically regulated. The physiologic pH range of the blood is fairly narrow and is maintained between 7.35-7.45 by various physiological complex homeostatic mechanisms, as graphically depicted in FIG. 3. Physiologic pH of the blood is bounded by an overly alkaline state (alkalosis) and an overly acidic state (acidosis). Alkalosis is the pathologic condition of blood pH being higher than 7.45 and can be the result of either an excess production of bicarbonate in the blood (metabolic alkalosis) or by decreased carbon dioxide levels in the blood (respiratory alkalosis). Alkalosis can cause various symptoms including confusion, nausea, vomiting, muscular twitching, and muscular spasms, which may need to be treated. Acidosis is the pathologic condition of blood pH being lower than 7.35 and can be the result of either an overproduction of an acid in the blood (metabolic acidosis), or by increased carbon dioxide levels in the blood resulting from compromised lung function or by depressed breathing or both (respiratory acidosis). Acidosis can cause various symptoms including confusion, headache, tachypnea (rapid breathing), and tachycardia (increased heart rate), which may need to be treated.

The lysosomotropic agent formulations (incl. ammonium chloride) provided by the present disclosure can be formulated in certain ways to fulfill the following requirements. First, the formulation can be configured to be orally administered. Second, the formulation can be configured to mitigate the risk of gastric side effects (upset stomach). Examples include where the lysosomotropic agent is coated by or packaged within an enteric coating or capsule, such as through use of an enteric coated softgel or capsule. Fourth, the formulation can be configured to mitigate the risk of unwanted blood pH variation outside of the 7.35-7.45 range by having a minimal ammonium chloride concentration variance in the blood and thus mitigate the risk of metabolic acidosis. This can be achieved by configuring the formulation to provide a sustained release of ammonium chloride in the duodenum/small intestine. Fifth, the formulation can result in increased lysosomal pH of the subject's cells and prohibit viral uncoating in lysosomes thereof, including uncoating of the SARS-CoV-2 virus, for example as shown in FIG. 2. It should be noted that the elevation of lysosomal pH by the formulation can be transient and occurs in response to the ammonium chloride administration. Once the administration and/or sustained release of ammonium chloride ends, the lysosomal pH can go back to the normal acidic state. The elevation of the lysosomal pH by the formulation is therefore only temporary and can be controlled by the administration and sustained release of the formulation.

The formulation can be configured as an oral unit dosage form with an enteric coating in order to release the lysosomotropic agent after the stomach, for example, in the upper tract of the intestine. The enteric-coated formulation can provide a sustained release dosage form. Unit dosage form examples including tablets, mini-tablets, pellets and granules or microspheres, usually filled into capsule shells. Enteric coatings can function by presenting a surface that is stable at the acidic pH found in the stomach, but which can break down at a higher pH (e.g., more alkaline pH). For example, the enteric coating will not dissolve in the gastric acids of the stomach (e.g., pH˜3), but will dissolve in the alkaline (pH 7-9) environment present in the small intestine. By preventing dissolution in the stomach, the enteric coating can also protect gastric mucosa from any irritating effects of one or more components of the formulation itself. When the formulation reaches the neutral or alkaline environment of the intestine, the coating can dissolve and components therein are available for absorption into the bloodstream.

There are various ways to make enteric formulations. Enteric coatings can include one or more fatty acids, waxes, shellac, polymers, and/or plant fibers, as known in the art. Certain materials that can be used to successfully formulate enteric coatings or enteric capsules include one or more of methyl acrylate-methacrylic acid copolymers, cellulose acetate phthalate, cellulose acetate succinate, hydroxypropyl methyl cellulose phthalate, hydroxypropyl methyl cellulose acetate succinate (hypromellose acetate succinate), polyvinyl acetate phthalate, methyl methacrylate-methacrylic acid copolymers, shellac, cellulose acetate trimellitate, sodium alginate, and zein. For example, an enteric coating aqueous solution (ethylcellulose, medium chain triglycerides [coconut], oleic acid, sodium alginate, stearic acid) can be used to form coated softgels.

There are various ways to make sustained release formulations. Such sustained release dosage forms include dosage forms configured to release liberate or liberate the lysosomotropic agent at a predetermined rate in order to maintain a constant concentration for a specific period of time with minimum side effects; e.g., deviation outside of a desired blood pH window. This can be achieved through a variety of formulations, including liposomes and drug-polymer conjugates (e.g., hydrogels). Sustained or modified-release dosages can permit the lysosomotropic agent to dissolve over time in order to be released slower and steadier into the bloodstream. A further advantage is that the sustained release formulation can be administered at less frequent intervals than immediate-release formulations of the same drug. The sustained release nature of the formulation can particularly advantageous for oral dose formulations. Timed release has several distinct variants such as sustained release where prolonged release is intended, pulse release, delayed release (e.g., to target different regions of the GI tract) etc. Sustained release not only it prolongs action of the lysosomotropic agent, but can maintain drug levels within the desired therapeutic window to avoid potentially hazardous peaks in drug concentration following ingestion or injection, to thereby maximize therapeutic efficiency.

One particular sustained release means that can be employed in the present formulations includes micro-encapsulation. Use of microspheres in formulation of the lysosomotropic agent can provide a predetermined dissolution profile. Microspheres can be used with enteric coatings or capsules, or the microspheres themselves can incorporate enteric coatings. In particular, the lysosomotropic agent can be coated around an inert core and layering with one or more insoluble substances to form microspheres, which can allow for consistent and replicable dissolution rates in a convenient format that can be mixed and matched with other components and/or pharmaceutical ingredients. It is further possible to make enteric capsules containing more than one type of microsphere, where at least some of the microspheres include the lysosomotropic agent.

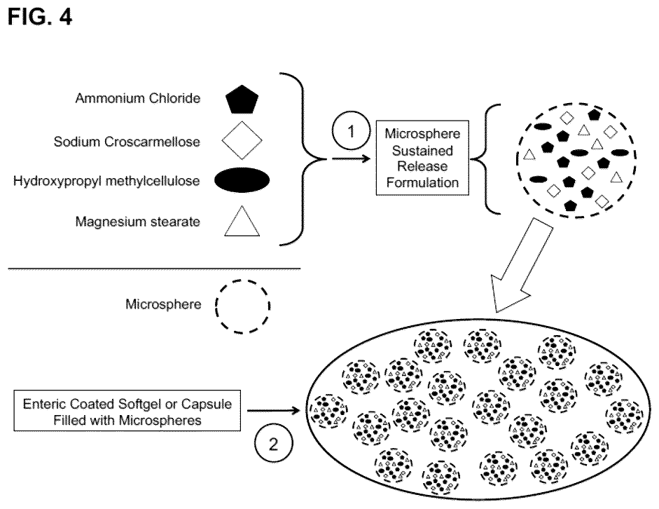

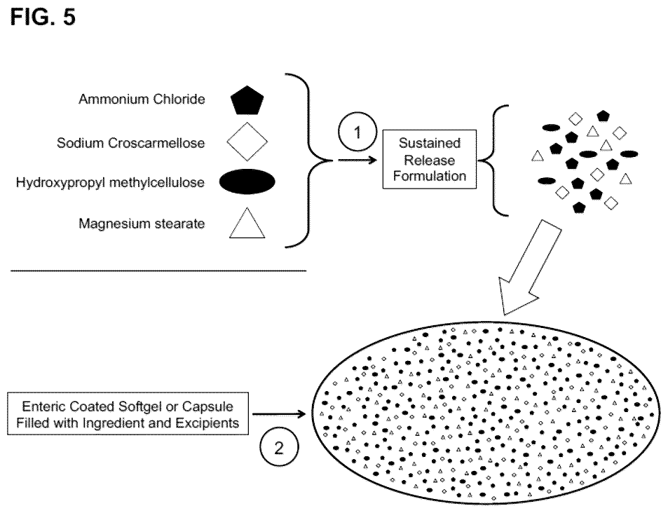

In certain embodiments, formulations provided by the present technology include a lysosomotropic agent as ammonium chloride, where the ammonium chloride is in the form of an enteric coated (gastro protective) softgel or capsule filled with microspheres including the components shown in FIG. 4, or with a mixture of the components shown in FIG. 5 not packaged as microspheres. As shown in FIGS. 4-5, the lysosomotropic agent as ammonium chloride can be formulated with excipients including sodium croscarmellose, hydroxypropyl methylcellulose, and magnesium stearate. Various unit dosage formulations can be made, including various oral unit dosage formulations. Regulatory status and function of the respective components are summarized below in Table 1.

TABLE 1

| | | | | Regu- | |
|---|---|---|---|---|---|
| | | | | latory | |
| # | Component | Quantity | | Status | Function |
| 1 | NH₄Cl | 10-1,000 | mg | GRAS | active ingredient, |
| | | | | | prohibits viral uncoating |
| | | | | | and release of viral RNA |
| 2 | sodium | 10-100 | mg | GRAS | excipients used for the |
| | croscarmellose | | | | sustained release of |
| 3 | hydroxypropyl | 10-100 | mg | GRAS | NH₄Cl |
| | methylcellulose | | | | |
| 4 | magnesium | 0.1-20 | mg | GRAS | |
| | stearate | | | | |

| | | | | |
|---|---|---|---|---|
| 5 | microspheres | 10-1000 | n/a | sustained release of |
| | | per softgel | | NH₄Cl |
| | | or capsule | | |
| 6 | enteric coated | 1-2/day | n/a | mitigation of gastric side |
| | softgel or | | | effects of NH₄Cl |
| | capsule | | | |

All ingredients of the ammonium chloride formulations of the present technology accordingly have GRAS status and can be used as ingredients/excipients in various oral dosage formulations, including formulations as dietary supplements and pharmaceutical formulations.

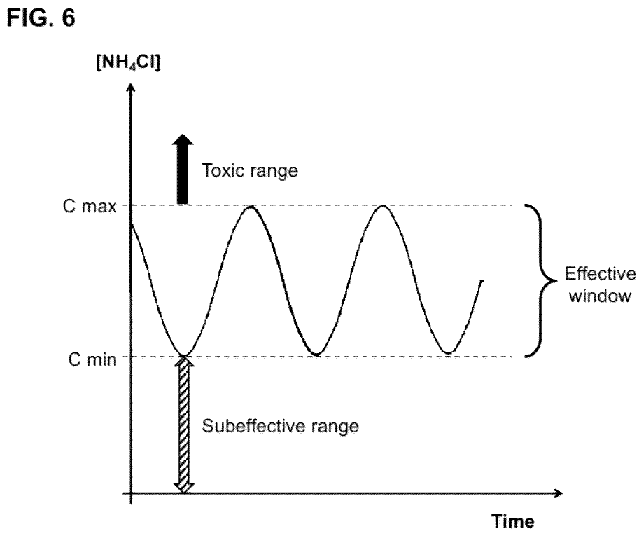

With reference now to FIG. 6, a graphical representation of blood concentration of ammonium chloride is shown following absorption from a sustained release formulation, where Cmain represents a minimum effective ammonium chloride blood concentration, Cmax represents a minimum toxic ammonium chloride blood concentration, and [Cmax−Cmin] represents an effective ammonium chloride blood concentration window.

In further embodiments of formulations of the lysosomotropic agent, vitamin D can be included in amounts of 1,000-4,000 International Units (“113”) per enteric coated softgel or capsule. Vitamin D can boost a subject's immune system and reduce inflammation, each of which can operate in conjunction with the lysosomotropic agent to militate against the effects of viral infection. Deficiency in vitamin D can also compound issues related to blood clots, where SARS-CoV-2 virus infection can result blood clots that can present serious issues in the subject's brain, heart, and lungs, for example. Formulations of the lysosomotropic agent can therefore include vitamin D to minimize such effects during and after administration.

The present technology further contemplates intravenous (IV) administration of formulations presented herein. Compared to per os (PO; i.e. oral) IV administration of ammonium chloride can have two major advantages. First, IV administration of ammonium chloride bypasses the stomach, and hence gastric side effects (e.g., upset stomach) related to PO administration of ammonium chloride are avoided. Second, the rate of IV administration of ammonium chloride can be easily adjusted, controlled, and monitored in order to avoid issues resulting from excessive administration of ammonium chloride, which can lead to toxic symptoms, including metabolic acidosis, hypokalemia, hypocalcemia, and hyperventilation, while maintaining the desired therapeutic effect.

The PO administration of ammonium chloride as a lysosomotropic agent must be done in such a way to also avoid the aforementioned side effects. In order to avoid the gastric side effects, the ammonium chloride can be formulated as a gastroprotective formulation (e.g., having an enteric coating) and be released from its gastroprotective formulation only after passing the stomach. The rate of ammonium chloride release from its gastroprotective formulation in the gut should be substantially constant in order to mimic the IV administration pharmacokinetic profile. The rate of ammonium chloride release from its gastroprotective formulation can be related to various specific formulation characteristics of its gastroprotective formulation including: (1) the quantity of ammonium chloride within its gastroprotective formulation; (2) the type of the excipients that are used in the gastroprotective formulation; (3) the quantity of each excipient used in the gastroprotective formulation; (4) the rate of ammonium chloride release from its gastroprotective formulation can be directly related with the rate of ammonium chloride absorption in the gut and the resulting ammonium chloride blood concentration.

To quantify the rate of ammonium chloride release from its gastroprotective formulation in vitro, it is possible to create various candidate gastroprotective ammonium chloride formulations with various quantities of ammonium chloride and one or more excipients. For each ammonium chloride candidate gastroprotective formulation, an in vitro dissolution study can be performed and a dissolution profile of ammonium chloride concentration over time can be plotted. One goal of such dissolution studies can be to establish an In-Vitro In-Vivo Correlation (IVIVC) as a way to assess the safety and efficacy of the ammonium chloride gastroprotective formulation. It is noted that dissolution methodology is established and well known, and one skilled in the art can readily ascertain a dissolution profile of ammonium chloride concentration over time and establish an IVIVC for various formulations, as presented herein. The U.S. Pharmacopeial Convention has further established certain dissolution measurement standard, where a specific Monograph exists for dissolution, USP <711>. Accordingly, it is possible to perform dissolution studies using the standards established by USP <711>, available at: [www.usp.org/sites/default/files/usp/document/harmonization/gen-method/stage_6_monograph_25_feb_2011.pdf]. Results of the gastroprotective, sustained release ammonium chloride dissolution studies can demonstrate the safety and efficacy of a respective formulation, as provided herein, and can show that the ammonium chloride release from the formulation and the resulting ammonium chloride absorption in the gut and overall pharmacokinetic profile can mimic the IV ammonium chloride administration pharmacokinetic profile.

Certain embodiments of the present technology provide formulations of the lysosomotropic agent (e.g., ammonium chloride) having surprising and unexpected dissolution characteristics, surprising and unexpected ammonium chloride absorption characteristics following oral administration, and/or surprising and unexpected pharmacokinetic performance. Such formulations can include an oral dose, such as doses provided in tablet form, that include a secosteroid, ammonium chloride, one or more long chain alcohols, a binder, a carboxylate salt, and one or more solvents. The formulation can have a coating system applied thereto, such as an enteric coating system.

Particular species of the formulation components can include the following materials. The secosteroid can include fat-soluble compounds that can increase intestinal absorption of minerals, such as calcium, magnesium, and phosphate, and which can provide other physiological effects. The secosteroid can include vitamin D, including vitamin D₃ (cholecalciferol) and vitamin D₂ (ergocalciferol). The secosteroid can be provided as a prohormone that is activated in vivo following administration of the formulation. The long chain alcohol can include a fatty alcohol, such as primary alcohols having carbon chains ranging from C₆-C₂₆. Certain examples of fatty alcohols include lauryl alcohol (C₁₂), myristyl alcohol (C₁₄), cetyl alcohol (C₁₆), stearyl alcohol (Cis), and arachidyl alcohol (C₂₀). The binder can include various water-soluble and polar polymers that can function as an emulsifier and a lubricant. The binder can also be configured to bind polar materials, due to its own polarity. A certain example of the binder includes polyvinylpyrrolidone. The carboxylate salt can include various salt forms of carboxylic acids, including salt forms of medium and long chain carboxylic acids. Examples of salt forms of long chain carboxylic acids composed of monovalent and divalent metals, such as sodium, calcium, magnesium, complexed with carboxylic acids having medium carbon chains ranging from C₆-C₁₂ and long carbon chains ranging from C₁₃-C₂₁. Certain examples include stearates, such as magnesium stearate. The solvent can include one or more polar solvents, including water and/or short carbon chain alcohols. Examples of short carbon chain alcohols include carbon chains ranging from C₁-C₅. A particular example of a short carbon chain alcohol includes ethanol. The coating system can include various coatings and polymers known in the pharmaceutical arts to provide an enteric coating so that substantially no dissolution of the formulation occurs in the stomach and the formulation provides sustained release of the lysosomotropic agent (e.g., ammonium chloride) after the formulation beings to dissolve at the small bowel.

Tablet formulations can include the components and amounts provided below in Table 2.

TABLE 2

| # | Component | Quantity per Tablet | |
|---|---|---|---|
| 1 | vitamin D (spray dried) | 1,000-4,000 | IU |
| 2 | NH₄Cl | 250-500 | mg |
| 3 | cetyl alcohol | 50-400 | mg |
| 4 | stearyl alcohol | 50-400 | mg |
| 5 | polyvinylpyrrolidone | 1-20 | mg |
| 6 | magnesium stearate | 2-20 | mg |
| 7 | ethanol | 0-100 | mg |
| 8 | purified water | 10-250 | mg |
| 9 | coating system | 0-30 | mg |

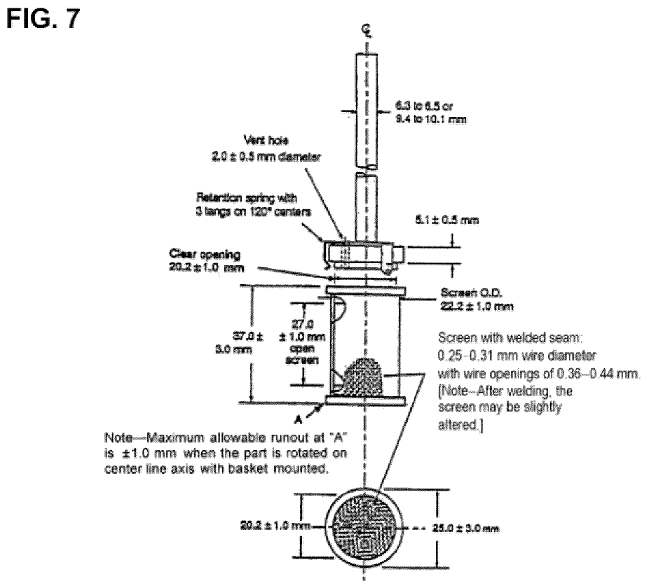

Dissolution profiles can be determined for formulation variants. The dissolution method can be performed as described in the aforementioned U.S. Pharmacopeial Convention Monograph USP <711>. The dissolution profile can range from 4 hours to 12 hours of full release of ammonium chloride content. Dissolution testing can employ a dissolution medium of 500-1000 mL H₂O, which may contain a small quantity of HCl or polysorbate 80. The dissolution testing apparatus can be configured as Apparatus 1 (basket apparatus) from U.S. Pharmacopeial Convention Monograph USP <711>, which can operate at a rotation between 50-100 rpm. A schematic of Apparatus 1 (basket apparatus) is shown in FIG. 7.

Apparatus 1 (basket apparatus) includes the following components: a vessel, which may be covered, made of glass or other inert, transparent material; a motor; a metallic drive shaft; and a cylindrical basket. The vessel is partially immersed in a suitable water bath of any convenient size or heated by a suitable device such as a heating jacket. The water bath or heating device permits holding the temperature inside the vessel at 37±0.5 degrees Celsius during the test and keeping the bath fluid in constant, smooth motion. No part of the assembly, including the environment in which the assembly is placed, contributes significant motion, agitation, or vibration beyond that due to the smoothly rotating stirring element. An apparatus that permits observation of the specimen and stirring element during the test is preferable. The vessel is cylindrical, with a hemispherical bottom and with one of the following dimensions and capacities: for a nominal capacity of 1 L, the height is 160 mm to 210 mm and its inside diameter is 98 mm to 106 mm; for a nominal capacity of 2 L, the height is 280 mm to 300 mm and its inside diameter is 98 mm to 106 mm; and for a nominal capacity of 4 L, the height is 280 mm to 300 mm and its inside diameter is 145 mm to 155 mm. Its sides are flanged at the top. A fitted cover may be used to retard evaporation. The shaft is positioned so that its axis is not more than 2 mm at any point from the vertical axis of the vessel and rotates smoothly and without significant wobble that could affect the results. A speed-regulating device is used that allows the shaft rotation speed to be selected and maintained at the specified rate given in the individual monograph, within ±4%.

Shaft and basket components of the stirring element are fabricated of stainless steel, type 316, or other inert material, to the specifications shown in FIG. 1. A basket having a gold coating of about 0.0001 inch (2.5 μm) thick may be used. A dosage unit is placed in a dry basket at the beginning of each test. The distance between the inside bottom of the vessel and the bottom of the basket is maintained at 25±2 mm during the test.

During testing, a sample of 10 ml can be withdrawn at each specified interval and filtered. To the sample, a mixture of previously neutralized to phenolphthalein, formaldehyde solution, and water is added. After 2 minutes, the sample can be titrated against 0.1 N NaOH using phenolphthalein as indicator. Each 1 ml of 0.1 N NaOH is equivalent to 5.349 mg of ammonium chloride. In this way, the content of ammonium chloride released at each specified interval is calculated.

Oral dosages based upon the formulations provided in Table 2 demonstrate beneficial and advantageous dissolution characteristics for intestinal uptake of ammonium chloride in militating against a clinical manifestation of infection by a virus in a subject.

In certain embodiments, formulations provided by the present technology include a lysosomotropic agent as ammonium chloride, where the ammonium chloride is in the form of an enteric coated (gastro protective) softgel or sustained-release tablet. The lysomotropic agent as ammonium chloride can be formulated with excipients including, but not limited to, at least one of stearic acid, acetyl alcohol, stearyl alcohol, polyvinylpyrrolidone, and magnesium stearate. The formulation can also include vitamin D. An example of the formulation makeup is summarized below in Table 3.

TABLE 3

| | Ingredients and excipients | | |
|---|---|---|---|
| # | (per 1 sustained-release tablet) | Quantity | |
| 1 | NH₄Cl | 500 | mg |
| 2 | Vitamin D3 (Cholecalciferol) | 2,000 | IU |
| 3 | Stearic acid | 250 | mg |
| 4 | Acetyl alcohol | 125 | mg |
| 5 | Stearyl alcohol | 125 | mg |
| 6 | Polyvinylpyrrolidone | 21.6 | mg |
| 7 | Magnesium stearate | 24.4 | mg |

Experimentation completed pursuant to the present disclosure shows that ammonium chloride reduced both intracellular and extracellular viral loads of SARS-CoV-2 and Influenza-A in cell culture experiments.

As an overview, the experimentation can include first determining limits of an NH₄Cl dose for cells used in the cell cultures to allow for adjusting an amount of NH₄Cl given to the cells to promote the viral statis effect without causing the cells to exhibit either alkalosis or acidosis. After the determination of the limits of the NH₄Cl dose for cells used in the cell cultures, the cell cultures can be infected with SARS-CoV-2 virus. After viral inoculation, and incubation for one hour, the NH₄Cl dose determined to be within the window of effective viral static effect without causing either alkalosis or acidosis can be added to the cultures. The intracellular viral load and the extracellular viral load of the cell cultures can be measured at 3, 6, 24, 48, and 72 hours post infection.

To determine the dose limits of NH₄Cl for cells to avoid alkalosis and acidosis, ammonium chloride dilutions 10:1 to 10:8 in culture medium can be added onto monolayers of the cells. Any microscopic changes in the cells after 5-days incubation are recorded. Cytotoxic effect on the cells used for the virus culture can be any change in morphology, growth, or susceptibility of the viral infected cells compared to control cells. The appropriate dosage of NH₄Cl provided to the cultures can be adjusted based on these results.

SARS-CoV-2 XBB.1.16 virus strain can be inoculated into confluent Vero E6 (ATCC, CRL-1586) cell monolayers and incubated for 1 hour with rocking. After inoculation, the samples are incubated in Dulbecco's Modified Eagle Medium (DMEM) supplemented with 2% heat-inactivated fetal bovine serum (FBS) at 37° C., and 5% CO₂. Influenza-A Wisconsin/67/2022 (H1N1) virus strain can be inoculated into Madin-Darby Canine Kidney (MDCK) cell monolayers (ATCC, CCL-34) in DMEM supplemented with 1 g/ml 1-1-tosylamide-2phenylethyl chloromethyl ketone (TPCK)-trypsin at 35° C., and 5% CO₂.

The supernatant (extracellular fluid) and a cellular pellet of the viral cultures are examined at five time-points (3 h, 6 h, 24 h, 48 h, and 72 h post infection) in both experimental (with NH₄Cl added) and the control (without addition of NH₄Cl) viral cultures. RT-PCR in each time point was conducted. Threshold cycles (Ct) derived from RT-PCR amplification plots can be calculated. The reduction of the viral RNA at the experimental cultures, compared to the viral RNA in control cultures indicates the viral-static activity of the NH₄Cl.

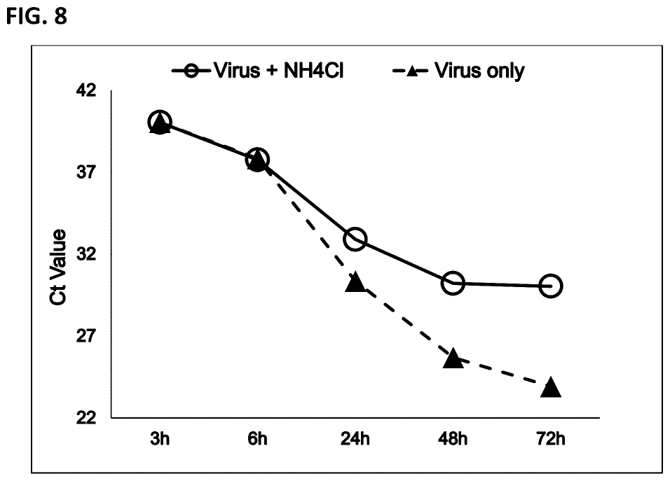

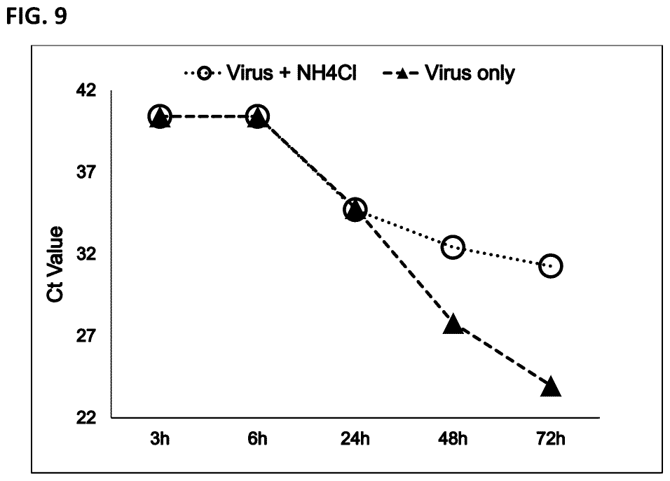

With reference FIGS. 8 and 9, for SARS-CoV-2, NH₄Cl treatment resulted in a reduction of intracellular viral load at 24, 48, and 72 hours post-infection, as shown below in Table 3 as well as FIG. 8, and a reduction of extracellular viral load at 48 and 72 hours post-infection, as shown below in Table 4 as well as FIG. 9. It can be seen that the lysosomotropic agent can be provided in an amount effective to increases the threshold cycle for detecting intracellular viral load by RT-PCR at 72 hours post-infection by at least 25%. For example, the difference between 23.92 Ct and 30.03 Ct relates to just over a 25% increase in threshold cycles (A of 23.92 Ct and 30.03 Ct is 6.11 Ct, where 6.11 Ct is a 25.5% increase in Ct over 23.92 Ct).

TABLE 4
SARS-CoV-2 Intracellular Viral Load

| | | |
|---|---|---|
| Time | Virus only | Virus + NH₄Cl |

| | | | |
|---|---|---|---|
| 3 h | 3 | 40.04 | 40.04 |
| 6 h | 6 | 37.83 | 37.75 |
| 24 h | 24 | 30.35 | 32.89 |
| 48 h | 48 | 25.68 | 30.21 |
| 72 h | 72 | 23.92 | 30.03 |

TABLE 5
SARS-CoV-2 Extracellular Viral Load

| | | |
|---|---|---|
| Time | Virus only | Virus + NH₄Cl |

| | | | |
|---|---|---|---|
| 3 h | 3 | 40.4 | 40.4 |
| 6 h | 6 | 40.4 | 40.4 |
| 24 h | 24 | 34.8 | 34.7 |
| 48 h | 48 | 27.79 | 32.41 |
| 72 h | 72 | 23.97 | 31.26 |

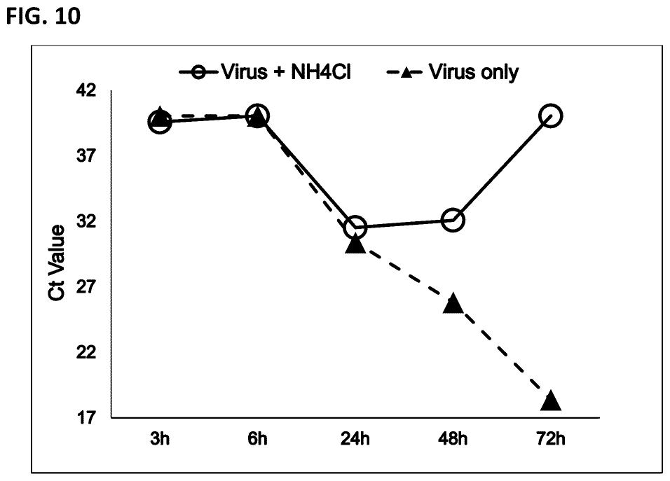

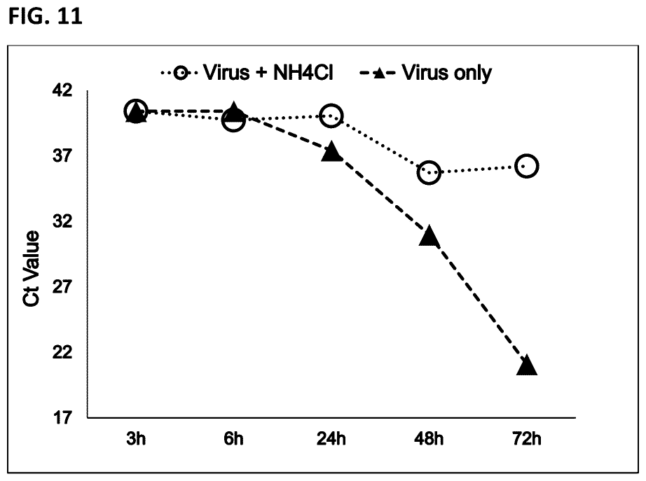

With reference FIGS. 10 and 11, for Influenza-A, NH₄Cl treatment resulted in a PG-5T reduction of intracellular viral load at 24, 48, and 72 hours post-infection, as shown below in Table 6 as well as FIG. 10, and a reduction of extracellular viral load at 48 and 72 hours post-infection, as shown below in Table 7 as well as FIG. 11. It can be seen that the lysosomotropic agent can be provided in an amount effective to increases the threshold cycle for detecting intracellular viral load by RT-PCR at 72 hours post-infection by at least 100%. For example, the difference between 23.92 Ct and 30.03 Ct relates to just over a 25% increase in threshold cycles (A of 18.38 Ct and 40.04 Ct is 21.66 Ct, where 21.66 Ct is more than a 100% increase in Ct over 18.38 Ct).

TABLE 6
Influenza-A Intracellular Viral Load

| | | |
|---|---|---|
| Time | Virus only | Virus + NH4Cl |

| | | | |
|---|---|---|---|
| 3 h | 3 | 40.04 | 39.57 |
| 6 h | 6 | 40.04 | 40.04 |
| 24 h | 24 | 30.38 | 31.52 |
| 48 h | 48 | 25.82 | 32.07 |
| 72 h | 72 | 18.38 | 40.04 |

TABLE 7
Influenza-A Extracellular Viral Load

| | | |
|---|---|---|
| Time | Virus only | Virus + NH4Cl |

| | | | |
|---|---|---|---|
| 3 h | 3 | 40.4 | 40.4 |
| 6 h | 6 | 40.4 | 39.72 |
| 24 h | 24 | 37.4 | 40.04 |
| 48 h | 48 | 30.97 | 35.7 |
| 72 h | 72 | 21.09 | 36.21 |

Cycle threshold (Ct) values were determined from quantitative RT-PCR (qPCR) tests to detect COVID-19 infection. The Ct value indicates the number of cycles needed for the qPCR fluorescent signal to exceed the background level, reflecting the amount of viral RNA present in a sample. Therefore, a lower Ct value indicates the presence of a higher viral load, meaning more viral RNA is present in the sample. Conversely, a higher Ct value indicates the presence of a lower viral load, meaning less viral RNA is present in the sample. These results presented the tables and graphs above demonstrate a significant and unexpected antiviral efficacy of NH₄Cl against SARS-CoV-2 virus.

### Examples

Examples of the present technology demonstrating effectiveness of a sustained-release ammonium chloride formulation in reducing the viral load of patients with COVID-19 or influenza are provided as follows.

Background: We estimated the effectiveness of a sustained-release dietary supplement formulation containing 500 mg ammonium chloride and 2,000 IU vitamin D (ACF; DIVIRNAM®) in reducing the viral load of patients with COVID-19 or influenza.

Methods: Eligible patients with COVID-19 or influenza were randomized to receive ACF twice daily or placebo (2,000 IU vitamin D/twice daily; DVF) for 10 days. Nasopharyngeal swab samples were collected at Day 1, Day 3-5 and Day 10-11 and tested for SARS-CoV-2 and influenza via RT-PCR. Cycle threshold (Ct) values were measured.

Results: Thirty-two patients were studied, 28 with COVID-19 and 4 with influenza. No patient developed severe disease, was hospitalized, or died. Sixteen patients received ACF and 16 VDF (mean age: 58.1 and 60.7 years, respectively; 68.8% and 25% with comorbidities, respectively). On Day 1, the mean Cts were 22.49 in ACF group and 21.01 in VDF group, on Day 3-5, the mean Cts were 33.20 and 30.82, respectively, and on Day 10-11, the mean Cts were 43.66 and 40.21, respectively. On Day 10-11 the adjusted mean difference was +3.12 cycles (95% confidence interval: 0.22-6.02; p-value=0.036). The Kaplan Meier analysis indicated faster clearance in the ACF group compared to the VDF group (p-value=0.016).

Conclusions: Our data indicate that ACF-receiving patients had a statistically significant reduction in viral load compared to placebo-receiving patients. This is attributed to the pharmacodynamic action of ammonium chloride and the pharmacokinetic properties of DIVIRNAM®. Larger studies are needed to further investigate the role of ACF in various RNA-viral infections.

Introduction: Respiratory viral infections are a leading cause of morbidity, mortality, and healthcare demand globally. The World Health Organization estimates that seasonal influenza (hereafter referred to as influenza) affects up to 20% of the global population and causes up to 650,000 deaths due to respiratory diseases annually. Almost six years after the emergence of severe acute respiratory syndrome coronavirus 2 (SARS-CoV-2) and the evolution of the coronavirus disease 2019 (COVID-19) pandemic, SARS-CoV-2 remains a significant cause of morbidity and mortality globally, mainly among older adults and individuals with comorbidities, due to the emergence of new variants that escape vaccine-derived immunity or immunity due to past infections. A meta-analysis of 21 studies conducted over two decades in seven high-income countries (France, Germany, Italy, Japan, Spain, the United Kingdom, and the United States) estimated that respiratory syncytial virus (RSV) will cause 5.7 million infections, 510,000 hospitalizations, and 37,000 deaths among adults ≥60 years in 2025 alone. In addition to respiratory viruses, other RNA viruses are major causes of morbidity and mortality globally such as hepatitis A, C, D and E viruses. Therefore, treatments to reduce the overall burden of RNA-viral infections are urgently needed.

Ammonium chloride (NH₄Cl), a lysosomotropic agent, demonstrates at specific concentrations a broad virostatic action through inhibition of virus uncoating and diminution or even inhibition of virus replication, and therefore delay of the progression of infection by various RNA viruses, including human and avian influenza A viruses, coronaviruses, and hepatitis A and C viruses. This effect is achieved through a temporary and reversible increase of the pH of intracellular lysosomes which in turn prevents the fusion between viruses and the lysosomes' membranes. In vitro studies have demonstrated the importance of endosomal acidification for SARS-CoV-2 entry and infection. Moreover, SARS-CoV-2-infected mice models have shown that ammonium chloride also possesses significant anti-SARS-CoV-2 activity, through inhibition of lysosomal acidification and therefore intracellular virus replication and alleviation of inflammation and infiltration in pulmonary tissues. In addition, a model using data from 51 countries or regions across Europe collected as of 26 Apr. 2020 confirmed the possible association between consumption of foods containing ammonium chloride and lower death rates from COVID-19. A randomized-controlled trial conducted in 2020 found that the time of recovery and SARS-CoV-2 loads were significantly reduced among COVID-19 patients who had received ammonium chloride (125 mg/5 ml daily per os) compared with the placebo group (60 patients in each group), by odds ratios of 1.8 [95% confidence interval (CI), 1.15-2.83; p-value=0.01] and 7.90 (95% CI, 1.62-14.17; p-value=0.014), respectively.

The current study aimed to estimate the effectiveness of a sustained-release dietary supplement formulation containing ammonium chloride and vitamin D (DIVIRNAM®) in reducing the viral loads of patients with COVID-19, influenza or RSV infection compared with the receipt of a vitamin D formulation only.

### Materials and Methods

Setting: This was a prospective, double-blind, randomized, comparative effectiveness study. The study was conducted from Sep. 1, 2024, to Apr. 30, 2025, at Sotiria Hospital for Respiratory and Thoracic Diseases and En Ygeia Clinic in Athens, Greece.

Study Population: Patients with influenza-like illness (ILI) or acute respiratory infection (ARI) who attended the Infectious Diseases Unit of Sotiria Hospital for Respiratory and Thoracic Diseases or the Internal Medicine Outpatient Clinic of En Ygeia Clinic during the study period were eligible for the study. Eligible patients were tested with a combo rapid antigen test for SARS-CoV-2, influenza A and B, and RSV (CorDX, Inc., San Diego, CA, USA). Inclusion criteria were an age ≥18 years, laboratory-confirmed SARS-CoV-2, influenza virus or RSV infection, and written informed consent. Patients were excluded if they were <18 years old, pregnant or lactating, had a hematologic malignancy, organ transplantation, a frailty score ≥5, symptoms for ≥3-5 days, or a documented allergy to ammonium chloride, vitamin D or any excipient of the administered formulations. Participation in the study was discontinued when one of the following occurred: intolerance of the administered formulations, non-compliance with study requirements or withdrawal of informed consent. The authors did not participate in patients' management decisions.

Laboratory testing: Nasopharyngeal swab samples were collected from each participant at three timepoints after diagnosis: Day 1 (baseline), Day 3-5 (intermediate), and Day 10-11 (final). All samples were transferred under ice-packed conditions to the Laboratory of the Research Group of Clinical Pharmacology and Pharmacogenomics at the Faculty of Pharmacy, School of Health Sciences, National and Kapodistrian University of Athens immediately after sampling, where they were kept at −20° C. for a maximum of three days until processing. Total RNA was extracted using the ZYBIO Nucleic Acid Extraction Kit (B-200-20) on the Zybio EXM3000 system, according to the manufacturer's protocol. Real-time reverse transcription polymerase chain reaction (RT-PCR) was performed using the Zybio SARS-CoV-2 & Influenza A/B Reagent Kit on the Zybio ZIP 96V thermocycler. This multiplex assay qualitatively detects SARS-CoV-2 RNA (N and S genes), influenza A (M gene), and influenza B (HA gene) through a one-step RT-PCR protocol. Cycle threshold (Ct) values were recorded for each sample. Each PCR run included both positive and negative controls, and at least one randomly selected sample from a prior run was included as a technical replicate for quality control purposes. For non-detects, Ct values were censored at the assay limit (45 cycles); any sample failing to return a valid Ct at any time-point was dropped entirely from that reagent's analysis. All PCR reactions were performed in a blinded manner with regard to group allocation, and blinding was maintained across laboratory technicians, case report form (CRF) handlers, and physicians until completion of the statistical analysis.

Study procedures: After baseline (Day 1) nasopharyngeal swab samples were collected, patients were randomly allocated in 1:1 ratio to receive either an enteric-coated sustained-release dietary supplement formulation containing 500 mg ammonium chloride, 2,000 IU vitamin D, and excipients (DIVIRNAM®) (ACF group) or a physically identical sustained-release dietary supplement formulation containing the same amount of vitamin D (2,000 IU) and the same excipients only (VDF group). ACF and VDF were administered twice daily (one tablet every 12 hours) for 10 consecutive days. Both formulations were visually indistinguishable and packaged in identical LOT-coded containers to ensure allocation concealment. The sustained-release ammonium chloride dietary supplement formulation has been described in the United States Patent Application Publication No.: 2022/0160757 (Priority Date: Nov. 20, 2020; Publication Date: May 26, 2022) and the International Patent Application Publication WO 2022/109393 (Priority Date: Nov. 20, 2020; Publication Date: May 27, 2022). In addition, ammonium chloride has been designated GRAS (Generally Recognized As Safe) status by the Food and Drug Administration of the United States (21CFR 184.1138), and it is already under registration in the European Union. Both ACF and VDF dietary supplement formulations have been notified to the Hellenic National Organization for Medicines (EOF).

Data collection: Patients were prospectively followed for a period of up to 30 days following laboratory-confirmed diagnosis. Data collection was conducted in real time by trained healthcare professionals through structured patient interviews at the time of enrolment and comprehensive review of medical records throughout the follow-up period. For each participant, a standardized CRF was utilized to systematically document the following information:
demographic data: age, sex
medical history: presence of comorbid conditions, including chronic cardiovascular disease, chronic pulmonary disease, diabetes mellitus, chronic kidney disease, chronic neuromuscular disorders, obesity, malignancy, and immunosuppression
infection and vaccination history: prior COVID-19 infections; COVID-19 vaccination status for the 2024-2025 season; influenza vaccination status for the 2024-2025 season
laboratory-confirmed diagnosis: SARS-CoV-2, influenza or RSV
clinical information: date of diagnosis, presenting symptoms, and subsequent clinical course, including hospitalization, need for supplemental oxygen, admission to an intensive care unit (ICU), and use of invasive mechanical ventilation
clinical outcome: status at timepoint 2 (Day 3-5), timepoint 3 (Day 10-11), and 30 days after diagnosis.

Definitions: ILI was defined as the sudden onset of at least one systemic symptom (fever, malaise, headache, myalgia) and at least one respiratory symptom (cough, sore throat, shortness of breath). ARI was defined as the sudden onset of at least one of the following symptoms: cough, sore throat, shortness of breath, and coryza. COVID-19 was defined as a case with symptoms compatible with COVID-19 and laboratory-confirmed SARS-CoV-2 infection. Influenza was defined as a case with symptoms compatible with influenza and a laboratory-confirmed influenza infection. RSV infection was defined as a case with symptoms compatible with RSV respiratory illness and laboratory-confirmed RSV infection. Mild illness was defined as the presence of various signs and symptoms (e.g., fever, cough, sore throat, malaise, headache, myalgia, nausea, vomiting, diarrhoea) but no shortness of breath, dyspnoea or abnormal findings on chest imaging. Severe illness was defined as increasing needs for supplemental oxygen, admission to ICU, invasive mechanical ventilation and/or death. Death was defined as 30-day crude mortality. Virus clearance was defined as a Ct value of ≥40.

Statistical analysis: Baseline characteristics were statistically compared using independent-samples t-tests or Mann-Whitney U tests for continuous variables, and Fisher's exact test for categorical variables. Differences in Ct values between groups were assessed using parametric [Welch's unequal-variance t-tests and analysis of covariance (ANCOVA)] and non-parametric (Mann-Whitney U tests, Hodges-Lehmann estimators and Cliff's δ effect sizes) approaches. The primary endpoint was analyzed using ANCOVA with baseline Ct as the covariate. Longitudinal trajectories were further evaluated using linear mixed-effects models for repeated measures (SPSS-MMRM), including fixed effects for group and visit, as well as their interaction. A random intercept was included for each subject, and the Kenward-Roger method was used to estimate the denominator degrees of freedom. Time-to-viral clearance was examined using Kaplan-Meier survival curves and discrete-time complementary log-log models. Robustness was assessed using HC3 heteroskedasticity-consistent errors, robust regression, permutation testing with Freedman-Lane residualisation, bootstrap resampling and leave-one-out diagnostics. All assumptions (normality, homoscedasticity and influence) were systematically checked. Analyses were conducted using IBM SPSS Statistics (version 26, IBM Corp., Armonk, NY, USA), Python (SciPy version 1.9) and GPower version 3.1.

### Results

A total of 32 participants were analyzed, with 16 assigned to the ACF group and 16 assigned to the VDF group. Table 8 shows the characteristics of patients stratified per group. The mean age was 58.1±17.6 years in the ACF group and 60.7±17.2 years in the VDF group. ACF group patients more frequently had at least one comorbidity compared with VDF-treated patients (68.8% versus 25.0%; p-value=0.02). Overall, chronic cardiovascular disease, chronic neuromuscular disease, and diabetes mellitus were the prevalent comorbidities among studied patients (6, 5, and 4 patients each).

TABLE 8
Baseline characteristics of participating
patients by treatment group.

| | ACF group | | VDF group | | p- |
|---|---|---|---|---|---|
| Characteristic | (n = 16) | | (n = 16) | | value |
| Mean age, years (±SD) | 58.1 | (±17.6) | 60.7 | (±17.2) | 0.24 |
| Median age, years | 59.5 | (20-81) | 57 | (36-95) | 0.91 |
| (range) | | | | | |
| Sex - Male | 4 | (25.0%) | 7 | (43.8%) | 0.44 |
| Sex - Female | 12 | (75.0%) | 9 | (56.2%) | |
| At least one | 11* | (68.8%) | 4** | (25.0%) | 0.02 |
| comorbidity | | | | | |
| Past SARS-CoV-2 | 16 | (100.0%) | 16 | (100.0%) | — |
| infection | | | | | |
| COVID-19 | 0 | (0.0%) | 0 | (0.0%) | — |
| vaccination*** | | | | | |
| Influenza | 0 | (0.0%) | 0 | (0.0%) | — |
| vaccination*** | | | | | |

ACF: ammonium chloride formulation; VDF: vitamin D formulation; SD: standard deviation; SARS-CoV-2: severe acute respiratory syndrome coronavirus 2; COVID-19: coronavirus disease 2019.
*11 ACF-treated patients had a total of 17 comorbidities: chronic cardiovascular disease: 5; chronic neuromuscular disease: 5; diabetes mellitus: 2; malignancy: 2; immunosuppression: 2; chronic renal disease: 1.
**4 VDF-treated patients had 4 comorbidities: diabetes mellitus: 2; chronic cardiovascular disease: 1; obesity: 1.
***for the 2024-2025 season.

Of the 32 patients studied, 28 had COVID-19 (14 in each group) and 4 had influenza A or B (2 in each group). No patient had RSV infection. All had a mild illness. No patient developed severe disease, was hospitalized, received oxygen, or died by 30 days after diagnosis. In general, the two groups did not differ regarding mean and median age, sex, past SARS-CoV-2 infection, and COVID-19 and influenza vaccination status for the 2024-2025 season. The only statistically significant difference was the prevalence of comorbidities, with the ACF group presenting a higher rate.

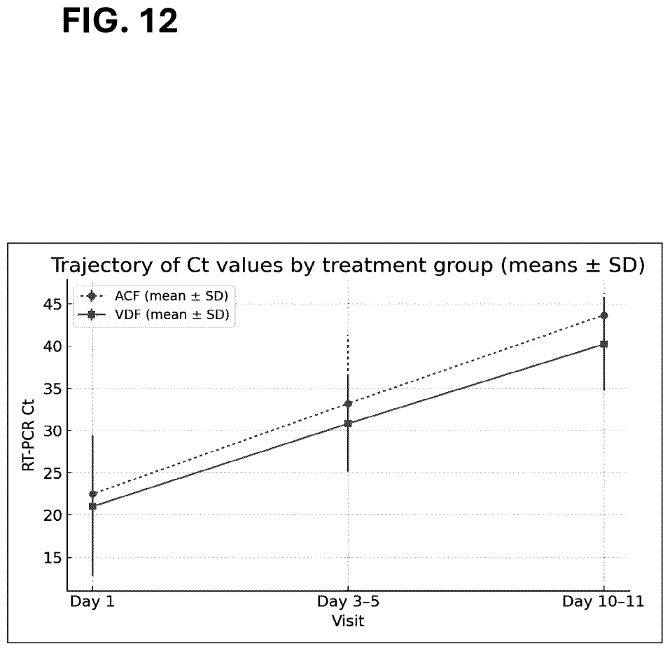

All 32 participants completed measurements on the three scheduled visits. FIG. 12 summarizes longitudinal Ct trajectories per group. On Day 1, the mean Ct values were 22.49±6.95 for the ACF group and 21.01±8.29 for the VDF group. By Day 3-5, the mean Ct increased to 33.20±8.10 in the ACF group and to 30.82±5.68 in the VDF group. At Day 10-11, the difference widened, with the ACF group showing a mean Ct of 43.66±2.57 compared to a mean of 40.21±5.47 in the VDF group. The increasing Ct values over time in both groups is consistent with declining viral load, with the greatest separation observed at Day 10-11 in favor of ACF. These preliminary findings motivated baseline-adjusted modeling and censoring-aware analyses presented below.

ANCOVA was conducted with treatment group as the main factor and baseline Ct as covariate. All 32 participants were included under the intention-to-treat principle. At Day 10-11, the adjusted mean difference (ACF−VDF) was +3.12 cycles (95% CI: 0.22-6.02), corresponding to a large, standardized effect size (Cohen's d≈0.81). The two-sided p-value was 0.036. In summary, the ACF formulation was associated with a statistically significant increase in Ct at Day 10-11 compared with VDF, indicating reduced viral load. Results from the MMRM analysis were consistent with the ANCOVA findings, confirming the longitudinal pattern of increasing Ct values and the treatment effect at Day 10-11 (Supplement 1).

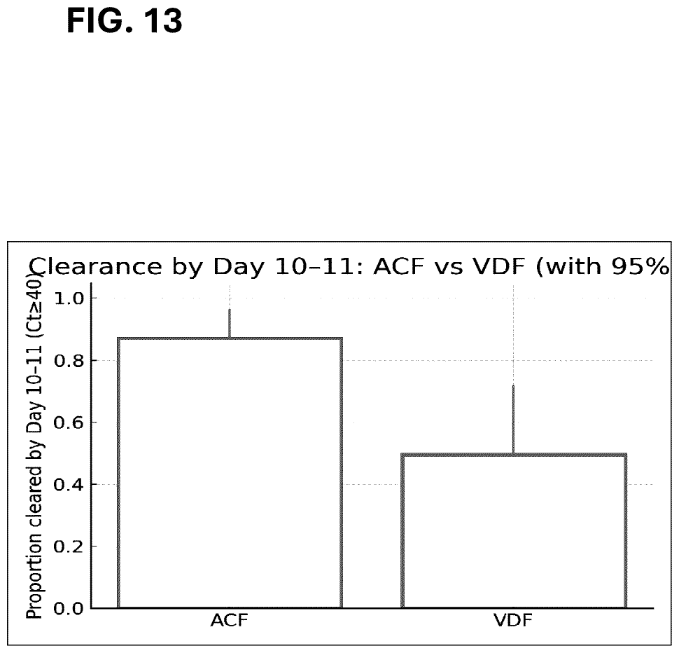

Time-to-viral clearance was assessed by defining the first participant visit at which Ct ≥40 as the clearance event, with participants not cleared by Day 10-11 censored at that visit. The Kaplan Meier analysis indicated faster clearance in the ACF group compared to VDF (log-rank p-value=0.016). By Day 10-11, clearance had been achieved in 14 of 16 ACF participants (87.5%) versus 8 of 16 VDF participants (50.0%). Effect measures derived at Day 10-11 showed a risk difference of +37.5 percentage points (95% CI: −8.0-68.5), a relative risk of 1.75 (95% CI: 1.04-2.95), and an odds ratio of 7.00 (95% CI: 1.18-41.36). These results are detailed in Table 9 and illustrated in FIG. 13. Notably, both ACF-treated patients with influenza noted early viral clearance, as noted by Ct values ≥40 at Day 5.

TABLE 9
Viral clearance by Day 10-11 and effect measures (ITT, Ct ≥ 40).

| Measure | Estimate | 95% CI |
|---|---|---|
| Cleared - ACF | 14/16 = 87.5% | [64.0%, 96.5%] (Wilson) |
| Cleared - VDF | 8/16 = 50.0% | [28.0%, 72.0%] (Wilson) |
| Risk difference (ACF − VDF) | 0.375 (37.5 pp) | [−0.080, 0.685] |
| Relative risk (ACF vs VDF) | 1.75 | [1.04, 2.95] |
| Odds ratio (ACF vs VDF) | 7.00 | [1.18, 41.36] |
| NNT | 2.7 | [1.5, ∞] |

ITT: Intention-to-Treat; Ct: cycle threshold; ACF: ammonium chloride formulation; VDF: vitamin D formulation; CI: confidence interval; NNT: Number Needed to Treat, where ITT: Intention-to-Treat, an analysis approach including all randomized participants in their originally assigned groups, regardless of adherence or protocol deviations; and NNT: Number Needed to Treat, the reciprocal of the absolute risk reduction, indicating how many patients must be treated with ACF instead of VDF for one additional patient to achieve viral clearance by Day 10-11.

To evaluate the robustness of the Day 10-11 findings, we conducted a series of sensitivity analyses using alternative specifications and inference frameworks. These included robust standard errors, robust regression, permutation testing, bootstrap resampling, and leave-one-out diagnostics. The sensitivity analyses demonstrate that the observed effect of ACF on Day 10-11 is robust to heteroskedasticity, outliers, and small-sample distribution concerns. These results are summarized in Supplement 2 and Supplement 3. Lastly, Table 10 compares the estimated differences between the ACF and VDF groups across multiple statistical approaches. Across all approaches, the Day 10-11 group effect consistently ranged from +2.6 to +3.1 cycles, with p-values around 0.03-0.04. The convergence of results across robust parametric and non-parametric frameworks increases confidence in the primary ANCOVA/MMRM signal.

TABLE 10
Estimated differences between the ACF and VDF
groups across multiple statistical approaches.

| | Effect size/ | |
|---|---|---|
| Analysis | Difference | Significance |
| ANCOVA (Day 10-11, | +3.12 Ct (Cohen's | p-value = 0.036 |
| adjusted) | d ≈ 0.81) | (nominal) |
| | | q = 0.098 (FDR) |
| MMRM (Day 10-11 LS- | +3.1 Ct (consistent | p-value ≈ 0.04 |
| means) | with ANCOVA) | (nominal) |
| | | q ≈ 0.10 (FDR) |
| Unadjusted Welch t-test | +3.45 Ct (Hedges' | p-value = 0.033 |
| (Day 10-11) | g ≈ 0.81) | |
| Non-parametric Hodges- | +3.45 Ct (median | 95% CI: 0.45-6.45 |
| Lehmann (Day 10-11) | difference) | |
| Time-to-clearance (Day | +37.5 percentage | 95% CI: −8.0-68.5 |
| 10-11, risk difference) | points | |
| Time-to-clearance (Day | OR: 7.00 | Significant |
| 10-11, OR) | 95% CI: 1.18-41.36 | |

ACF: ammonium chloride formulation;
VDF: vitamin D formulation;
ANCOVA: analysis of covariance;
Ct: cycle threshold;
FDR: False Discovery Rate;
q: FDR adjusted p-value;
MMRM: Mixed-Effects Model for Repeated Measures;
LS: Least Squares means;
CI: confidence interval;
OR: odds ratio
q: adjusted p-value after controlling for the false discovery rate (FDR).
FDR: False Discovery Rate, a multiple-comparison correction method (e.g., Benjamini-Hochberg) controlling the expected proportion of false positives.
MMRM: Mixed-Effects Model for Repeated Measures, a longitudinal model that accounts for within-subject correlations across timepoints.
LS: Least Squares means (also called Estimated Marginal Means), model-adjusted group averages derived from MMRM rather than raw arithmetic means.

### Discussion

The present double-blind, randomized study indicates that patients who received a sustained-release dietary supplement formulation containing 500 mg ammonium chloride and 2,000 IU vitamin D (DIVIRNAM®) twice daily had statistically significant lower viral loads compared to placebo-receiving patients. The largest consistent differences in Cts between the ACF and VDF groups were recorded on Day 10-11 and were confirmed by multiple statistical approaches. Moreover, the time-to-clearance analysis demonstrated that virus clearance occurred more rapidly and more frequently in the ACF group compared with the VDF group, which provides further evidence of antiviral effectiveness. Notably, despite a higher prevalence of baseline comorbidities, ACF-treated patients achieved faster and more frequent viral clearance, suggesting that the treatment effect was not simply due to baseline differences; however, this observation should be interpreted with caution due to the small sample size. To our knowledge, there is only one study published so far, which showed a significant reduction of the time of recovery as well as of SARS-CoV-2 loads among hospitalized COVID-19 patients who received ammonium chloride, but not via a sustained-release ammonium chloride formulation. To our knowledge, there is no published data regarding the administration of ammonium chloride to patients with influenza or other respiratory viruses.

Monitoring of viral loads through PCR has become the standard of care for estimating the effectiveness of treatments for various viral infections. In our study, the gradual but consistent increase in Ct values observed among patients who received the ACF formulation is attributed to the combination of the pharmacodynamic action of ammonium chloride along with the pharmacokinetic properties of the ACF sustained-release formulation. The ACF dietary supplement formulation incorporates a 12-hour release mechanism to support prolonged immunomodulatory activity, therefore enabling the gradual and durable suppression of virus replication compared with the VDF formulation that lacks ammonium chloride. Studies are needed to investigate the effects of ammonium chloride on viral load of other RNA viruses that constitute public health threats, such as influenza A and H5N1.

In our study, both patients with influenza who received the ammonium chloride formulation noted early viral clearance, as highlighted by Ct values ≥40 at Day 5. This pattern was not observed in the VDF-receiving influenza cases. While the number of patients with influenza was too small for statistical analysis, the trend could suggest a potentially enhanced antiviral effect of the ACF on influenza virus. Given the very small number of influenza patients, this observation is descriptive and hypothesis-generating rather than confirmatory. This finding may indicate a potentially enhanced antiviral effect of the ACF on influenza virus and can be elucidated through targeted studies in patients with influenza, particularly in light of shifting public health priorities, where COVID-19 concern is gradually subsiding and influenza remains a recurrent burden with significant clinical impact.

Prospective studies have demonstrated that vitamin D supplementation has a protective effect on ILL. In addition, vitamin D supplementation, particularly when administered early in the course of COVID-19, ameliorated its clinical course and prognosis. The positive effects of vitamin D are mediated by enhancing the innate antiviral immune response, facilitating the induction of antimicrobial peptides/autophagy, and modulating the host reactive hyperinflammatory phase during COVID-19. However, mixed results from other trials led to a lack of consensus on optimal dosing and timing of vitamin D supplementation in COVID-19. In our study, we assumed that vitamin D served as a booster for the intrinsic benefits conferred by ammonium chloride to the ACF-receiving patients. Both study formulations contained the same dose of vitamin D (2,000 IU twice daily); therefore, any between-group differences observed can be attributed to the presence or absence of ammonium chloride rather than to vitamin D supplementation.

The main strength of the present study is its prospective, double-blind, randomization design. This enabled the repeated nasopharyngeal swab sampling in predefined time points along with the study of the kinetics of Ct values. In addition, data about the clinical course and outcome were collected prospectively.

The main limitation is the small number of study participants, which explains the wide CIs, and which did not allow the study of ACF on viral loads by virus, age groups, comorbidities, and settings (e.g. hospital versus ambulatory healthcare settings). Another limitation is that the precise time from the onset of symptoms to treatment was not available; nevertheless, all studied patients were enrolled 3-5 days after the onset of symptoms. The fact that Ct values concern viral nucleic acid and do not necessarily correspond to infectious virus should also be considered. Lastly, heterogeneity was present in our study but was adequately addressed through robust inference, and raised confidence regarding the primary ANCOVA results.

Additionally, two supplementary analyses were performed to address censoring, multiplicity, and statistical power. First, a Tobit regression model (censored normal) was applied to account for right-censoring at the PCR assay limit (Ct=45). The estimated latent-scale effect was +6.40 cycles (95% CI: −4.47 to 17.27, p=0.248), while the average marginal effect on the observed scale was +2.55 cycles (95% CI: −2.20 to 8.94). These results aligned directionally with the ANCOVA findings but were not statistically significant, mainly because they reflected the high proportion of censored observations (20/32 participants). Second, a post hoc power analysis was conducted based on the observed ANCOVA effect (A=+3.12 Ct, Cohen's d≈0.80). With N=32 (16 per arm), the achieved power was approximately 60% at α=0.05 (two-sided). Prospective sample size estimates indicated that ˜17 participants per arm would be required for 80% power and ˜23 per arm for 90% power, assuming a true effect of ˜3 cycles, while smaller effects (2.0-2.5 cycles) would require 25-50 participants per arm.

In conclusion, the findings of the present study indicate that a sustained-release ammonium chloride formulation (DIVIRNAM®) administered twice daily significantly reduced the viral load of patients with COVID-19 or influenza compared to placebo-receiving patients. This is attributed to the pharmacodynamic action of ammonium chloride and the pharmacokinetic properties of DIVIRNAM®. Larger studies are needed to further investigate the effectiveness of DIVIRNAM® in various RNA-viral infections, patient populations, and healthcare or non-healthcare settings.

The following glossary of statistical terms and abbreviations applies to the preceding example.
[symbol] ANCOVA (Analysis of Covariance): A general linear model that combines ANOVA and regression, used here to compare cycle threshold (Ct) values between groups while adjusting for baseline Ct.
[symbol] ARR (Absolute Risk Reduction): The difference in event rates between two groups; used to calculate the NNT.
[symbol] Bootstrap (Percentile CI): A resampling method that estimates confidence intervals by repeatedly drawing samples with replacement from the data.
[symbol] CI (Confidence Interval): A statistical interval estimate that provides a range of values within which the true parameter is expected to lie with a specified probability (commonly 95%).
[symbol] Cliff's δ (delta): A non-parametric effect size measure representing the probability that a randomly selected value from one group exceeds a randomly selected value from another group.
[symbol] Cohen's d/Hedges' g: Standardized effect size measures representing the difference between two means expressed in units of standard deviation.
[symbol] Ct (Cycle Threshold): The number of PCR cycles required for the fluorescent signal to cross the detection threshold; inversely proportional to the viral load.
[symbol] FDR (False Discovery Rate): A multiple-comparison correction method (e.g., Benjamini-Hochberg) that controls the expected proportion of false positives among results declared significant.
[symbol] HC3 robust SE (Heteroskedasticity-Consistent Standard Errors, type 3): A variance estimator that adjusts for heteroskedasticity and small-sample bias in OLS regression.
[symbol] Hodges-Lehmann Estimator: A non-parametric estimator of the median difference between two groups, robust to non-normal distributions.
[symbol] ITT (Intention-to-Treat): An analysis principle in which all randomized participants are analyzed in their originally assigned groups, regardless of adherence or protocol deviations.
[symbol] Kaplan-Meier Curve: A survival analysis method used to estimate the probability of an event (here, viral clearance) over time.
[symbol] LS (Least Squares Means or Estimated Marginal Means): Model-adjusted group averages derived from ANCOVA or MMRM, representing expected values after accounting for covariates, not simple arithmetic means.
[symbol] MMRM (Mixed-Effects Model for Repeated Measures): A longitudinal statistical model that accounts for correlations within subjects across repeated timepoints, including both fixed and random effects.
[symbol] NNT (Number Needed to Treat): The reciprocal of the absolute risk reduction; indicates how many patients must be treated with ACF instead of VDF for one additional patient to achieve viral clearance by Day 10-11.
[symbol] OLS (Ordinary Least Squares): The standard estimation method for linear regression and ANCOVA, minimizing the sum of squared residuals to obtain parameter estimates.
[symbol] OR (Odds Ratio): A measure of association representing the odds of an event occurring in one group compared to another.
[symbol] Permutation Test (Freedman-Lane): A non-parametric resampling method for testing significance by permuting residuals, robust to distributional assumptions.
[symbol] p-value: The probability of observing results as extreme as (or more extreme than) those obtained, under the null hypothesis of no difference.
[symbol] q-value: The false discovery rate (FDR)-adjusted p-value, representing the minimum FDR at which a particular test result would be considered significant.
[symbol] RR (Relative Risk): The ratio of the probability of an event in the treatment group to that in the control group.

Another example of the present technology includes the following demonstration of the safety and tolerability of prolonged high-dose sustained-release ammonium chloride formulation administration in healthy adults.

Background: To evaluate the safety and tolerability of a sustained-release dietary supplement formulation containing 500 mg ammonium chloride and 2,000 IU vitamin D per tablet (DIVIRNAM®) administered three times daily for 30 days instead of the standard administration (twice daily for 10 consecutive days).

Methods: Fifteen individuals (9 females, 6 males; age range: 23-63 years) were studied. Participants were healthy adults (≥18 years) tested negative for SARS-CoV-2, influenza A/B, and respiratory syncytial virus. Participants self-recorded any adverse event daily throughout the 30-day supplementation period and underwent laboratory testing at baseline and immediately after the 30 days supplementation for hematologic and inflammatory parameters, metabolic, hepatic, and renal markers; electrolytes; venous blood gases; and 25-hydroxyvitamin D (“vitamin D”) serum levels.

Results: The supplement was well tolerated. All participants completed the supplementation. Eleven participants (73.34%) developed mild, self-limited reactions, mainly gastrointestinal symptoms (soft stools and/or bloating) and increased thirst, which did not necessitate medical intervention or study discontinuation. No serious adverse events occurred. The mean serum 25(OH)D levels increased by 40.8% after the 30-day supplementation (p-value=0.0016), resulting in 14 out of 15 participants achieving sufficient vitamin D levels by the end of the study. While a few laboratory parameters increased significantly by the end of the study period, all said parameters remained within the normal reference ranges.

Conclusions: Our findings indicate that DIVIRNAM® is safe and well tolerated even when administered at the elevated dose of three tablets daily for a prolonged period of 30 consecutive days.

Introduction: Ammonium chloride (NH₄Cl) is a broad-spectrum virostatic agent. The action of ammonium chloride is achieved through a temporary, reversible increase of the pH of intracellular lysosomes, resulting in inhibition of virus uncoating, prevention of fusion between viruses and lysosomal membranes, and subsequent reduction or inhibition of intracellular replication of several RNA viruses, including coronaviruses, influenza viruses, and hepatitis A and C viruses. Mice models have shown that ammonium chloride has significant anti-SARS-CoV-2 activity while one study demonstrated that patients with COVID-19 treated with ammonium chloride (at a dose of 125 mg/5 ml daily per os) recovered significantly earlier compared with untreated COVID-19 patients by an odds ratio of 1.8 [95% confidence interval (CI): 1.15-2.83; p-value=0.01]. In our previous work we found a statistically significant reduction in viral load of patients with COVID-19 or influenza who had received a sustained-release oral dietary supplement containing 500 mg ammonium chloride and 2,000 IU vitamin D (DIVIRNAM®) twice daily for 10 consecutive days, compared to placebo-receiving patients.

The current study aimed to evaluate the safety and tolerability of prolonged administration of high-dose of a sustained-release oral supplement containing 500 mg ammonium chloride and 2,000 IU vitamin D (DIVIRNAM®) in healthy adults.

### Materials and Methods

Setting: This was a prospective, single-arm, open-label safety and tolerability study. The study was conducted at Sotiria Hospital for Respiratory and Thoracic Diseases in Athens, Greece, between Apr. 12, 2025, and Jun. 28, 2025.

Study Population: Fifteen individuals were enrolled in the study, including healthcare personnel from Sotiria General Hospital and healthy adult volunteers from the general population who met the eligibility criteria. All eligible individuals were tested with a combo rapid antigen test for severe acute respiratory syndrome-coronavirus 2 (SARS-CoV-2), influenza A and B, and respiratory syncytial virus (RSV) (CorDX, Inc., San Diego, CA, United States). Eligible participants were adults aged 18 years or older with a negative rapid antigen test for SARS-CoV-2, influenza A and B, and RSV. All participants were required to be free from any diagnosed systemic disease at the time of enrolment. Exclusion criteria included age under 18 years, pregnancy or lactation, presence of any active infection, documented hypersensitivity or known allergy to any component of the investigational supplement, and failure or unwillingness to provide written informed consent. Participation in the study was discontinued when one of the following occurred: intolerance of the supplement formulation, non-compliance with study requirements or withdrawal of informed consent.

Laboratory testing: Ten ml of venous blood was drawn from each participant at two timepoints: Day 0 (baseline, before supplementation) and Day 31 (one day after the end of the 30-day supplementation). The following laboratory tests were performed at each timepoint:
[symbol] hematologic and inflammatory parameters [red blood cells (RBC), red cell distribution width (RCDW), hemoglobin, hematocrit, mean corpuscular volume (MCV), mean corpuscular hemoglobin (MCH), mean corpuscular hemoglobin concentration (MCHC), while blood cells (WBC), differential counts, platelets]
[symbol] metabolic, hepatic, and renal markers [glucose, urea, creatinine, aspartate aminotransferase (AST), alanine aminotransferase (ALT), alkaline phosphatase (ALP), total and direct bilirubin, total proteins, albumin]
[symbol] serum electrolytes (e.g., sodium, potassium, calcium, chloride)
[symbol] venous blood pH
[symbol] serum 25-hydroxyvitamin D (25(OH)D)

Venous blood gas analysis was conducted immediately post phlebotomy at the Intensive Care Unit of the Sotiria hospital. Subsequently, specimens were transported within 30 to 60 minutes from collection to the clinical laboratory En Ygeia for further laboratory analysis. Transport was carried out using certified biological specimen containers equipped with ice packs to maintain appropriate temperature conditions. All laboratory analyses were performed on the same day as sample collection.

Study procedures: At baseline (Day 0), peripheral venous blood samples were collected from all participants. Each individual then received a 30-day supply of a sustained-release dietary supplement formulation (DIVIRNAM®), consisting of 90 tablets, each containing 500 mg of ammonium chloride and 2,000 IU of vitamin D₃. Participants were provided with detailed instructions for oral administration of the supplement at a dosage of one tablet every 8 hours (three tablets daily). This administration schedule represents a 50% increase in daily dosage and a 300% extension of total duration compared to the standard recommended schedule of 2 tablets daily for 10 days. The sustained-release ammonium chloride dietary supplement formulation has been described in the United States Patent Application Publication No.: 2022/0160757 (Priority Date: Nov. 20, 2020; Publication Date: May 26, 2022) and the International Patent Application Publication WO 2022/109393 (Priority Date: Nov. 20, 2020; Publication Date: May 27, 2022). Ammonium chloride has been designated GRAS (Generally Recognized As Safe) status by the United States Food and Drug Administration (21CFR 184.1138), and it is already registered in the European Union. In addition, DIVIRNAM® has been notified by the Hellenic National Organization for Medicines (EOF).

Data collection: The following data were collected at the time of enrolment using one case report form per participant: age, sex, underling conditions/comorbidities, and concomitant medications. A standardized diary was administered to participants for daily self-recording of any adverse events and adherence to the recommended schedule (3 tablets daily for 30 consecutive days). After completion of the 30-day regimen, participants were asked about their willingness to repeat the supplementation at the standard recommended dosage. Participants were also contacted on Day 15, Day 30 and one week after the completion of the 30-day supplementation for adverse events. Adverse events were classified as mild (no interference with daily activity), moderate (some interference with daily activity), severe (prevention of daily activity) or potentially life-threatening (emergency department visit and/or hospitalization).

Statistical analysis: The primary endpoint of the study was the onset of adverse events, at any time during, or at the end of the 30-day supplementation period, while secondary endpoints included changes in laboratory parameters between Day 0 and Day 30. Normality of continuous variables was assessed with the Shapiro-Wilk test. For normally distributed data, paired t-tests were applied to compare baseline and end-of-study values, while non-normally distributed data were analyzed with the Wilcoxon signed-rank test. Statistical significance was set at p-value≤0.05 (two-tailed). All analyses were conducted in SPSS (version 30).

### Results

Fifteen individuals completed the 30-day regimen and were included in the analysis. Participants had a mean age of 37 years (range: 23-63 years); there were 9 females and 6 males. Table 1 shows the reported tolerability, adherence to, and willingness to repeat the standard 10-day supplementation with sustained-release ammonium chloride. The formulation was well tolerated at the dose of 3 tablets daily. Eleven participants (73.34%) developed mild, self-limited reactions, mainly gastrointestinal symptoms (soft stools and/or bloating) and increased thirst. The symptoms occurred during the first few days of intake. Overall adherence was high, with 10 of 15 participants (66.67%) reporting a median of 5 missed doses (range: 1-12), including one participant who developed a confirmed gastroenteritis episode unrelated to the supplement, which led to discontinuation of supplementation for four consecutive days (12 missed doses) before voluntarily resuming and completing the protocol. No serious adverse events occurred. Overall, all but one participant (93.34%) reported definite or probable willingness to repeat a 30-day supplementation course. Lastly, two participants reported a reduction in joint pain during the supplementation period, while one participant described increased energy levels and improved evening stamina.

Raw participant-level data and statistical analyses per laboratory parameter are available in the supplementary Table 11.

TABLE 11
Reported adverse events, adherence to, and willingness to repeat a course
of supplementation* following the initial 30-day intake period.

| | | | Missed | | |
|---|---|---|---|---|---|
| Partic- | Age/ | Adverse | doses | Willingness | |
| ipant | sex | events | (n) | to repeat | Notes |
| 1 | 36 | soft stools; | 2 | definitely | reduction in |
| | F | bloating | | yes | joint pain |
| 2 | 36 | bloating | 0 | definitely | none |
| | M | | | yes | |
| 3 | 23 | none | 3 | probably | mild iron- |
| | F | | | yes | deficiency |
| | | | | | anemia |
| 4 | 30 | increased thirst; | 6 | definitely | on short-term |
| | F | urinary | | yes | analgesics |
| | | frequency | | | |
| 5 | 55 | soft stools | 0 | probably | on rosuvastatin |
| | M | | | yes | 20 mg daily |
| 6 | 41 | none | 0 | not sure | increased |
| | F | | | | energy levels |
| 7 | 45 | soft stools | 5 | probably | none |
| | F | | | yes | |
| 8 | 27 | Increased thirst | 0 | probably | none |
| | F | | | yes | |
| 9 | 32 | soft stools; | 1-2 | probably | reduction in |
| | M | increased thirst | | yes | joint pain |
| 10 | 38 | upset stomach; | 5 | probably | none |
| | M | increased thirst | | yes | |
| 11 | 48 | bloating | 2 | probably | none |
| | F | | | yes | |
| 12 | 32 | soft stools; | 10 | probably | none |
| | M | fatigue; | | yes | |
| | | increased thirst | | | |
| 13 | 25 | none | 5 | probably | none |
| | F | | | yes | |
| 14 | 28 | bloating | 0 | definitely | none |
| | M | | | yes | |
| 15 | 63 | none | 12 | probably | GE on day 3; |
| | F | | | yes | paused 4 days; |
| | | | | | on rosuvastatin |
| | | | | | 10 mg daily |

GE: gastroenteritis
*sustained-release oral supplementation containing ammonium chloride and vitamin D (DIVIRNAM ®) at a dose of 2 tablets per day for 10 days

Table 12 summarizes the laboratory parameters of the participants in Day 0 and Day 30 while.

TABLE 12
Laboratory parameters in baseline (Day 0) and end of intake (Day 30)

| | Mean | Mean | | | | | |
|---|---|---|---|---|---|---|---|
| | (SD) | (SD) | Mean | | | Normal | |
| | in Day | in Day | difference | % | | reference | |
| Parameter | 0 | 30 | (95% CI) | difference | p-value | ranges/units | |
| Biochemical | | | | | | | |
| Glucose | 85 | 86 | +0.67 | 0.80% | NS | 65-110 | mg/dL |
| | (10.5) | (10.8) | (−4.4 to +6.4) | | | | |
| Urea | 34 | 32 | −1.6 | −4.7% | NS | 10-50 | mg/dL |
| | (9.7) | (7.1) | (−5.3 to +3.7) | | | | |
| Uric acid | 3.8 | 3.7 | −0.15 | −3.9% | NS | 2.5-6 | mg/dL |
| | (1.5) | (1.3) | (−0.6 to +0.4) | | | | |
| Creatinine | 0.90 | 0.89 | −0.03 | −3.4% | NS | 0.6-1.3 | mg/dL |
| | (0.22) | (0.25) | (−0.09 to +0.07) | | | | |
| AST | 20.5 | 19.5 | −1.4 | −6.7% | NS | 5-45 | U/L |
| | (3.4) | (2.8) | (−2.8 to +0.8) | | | | |
| ALT | 17.1 | 17.2 | +0.2 | 1.10% | NS | 7-40 | U/L |
| | (5.9) | (5.7) | (−2.0 to +2.2) | | | | |
| ALP | 60.3 | 67.3 | +8.53 | 14.20% | <0.001 | 42-98 | U/L |
| | (21.4) | (23.1) | (−1.2 to +15.2) | | | | |
| Total bilirubin | 0.6 | 0.6 | 0.0 | 0% | NS | 0.2-1.2 | mg/dL |
| | (0.2) | (0.1) | (−0.06 to +0.06) | | | | |
| Direct bilirubin | 0.13 | 0.12 | −0.01 | −7.7% | NS | <0.3 | mg/dL |
| | (0.04) | (0.04) | (−0.03 to +0.01) | | | | |
| Total proteins | 7.4 | 7.4 | 0.06 | 0.80% | NS | 6.3-8.6 | g/dL |
| | (0.4) | (0.6) | (−0.3 to +0.3) | | | | |
| Albumin | 4.6 | 4.4 | −0.13 | −2.8% | 0.019 | 3.5-5.0 | g/dL |
| | (0.3) | (0.2) | (−0.3 to −0.1) | | | | |
| Electrolytes | | | | | | | |
| Sodium | 139.1 | 139.5 | +0.5 | 0.40% | NS | 135-145 | mEq/L |
| | (1.2) | (2.0) | (−0.4 to +1.6) | | | | |
| Potassium | 4.4 | 4.2 | −0.1 | −4.5% | 0.023 | 3.5-5.1 | mEq/L |
| | (0.2) | (0.2) | (−0.2 to 0.0) | | | | |
| Calcium | 9.61 | 9.53 | −0.08 | −0.8% | NS | 8.2-10.4 | mg/dL |
| | (0.3) | (0.2) | (−0.2 to 0.0) | | | | |
| Chloride | 106.2 | 106.3 | +0.13 | 0.10% | NS | 98-108 | mmol/L |
| | (1.5) | (1.9) | (−0.9 to +1.9) | | | | |
| Hematological | | | | | | | |
| WBC | 6.39 | 6.45 | +0.06 | 0.90% | NS | 4.0-10.0 × | |
| | (1.4) | (1.5) | (−0.5 to +0.7) | | | 10³/μL | |
| RBC | 4.87 | 4.88 | 0.01 | 0.20% | NS | 3.6-5.0 × | |
| | (1.0) | (1.0) | (−0.1 to +0.1) | | | 10⁶/μL | |
| HGB | 13.7 | 13.5 | −0.27 | −2% | NS | 12-16 | g/dL |
| | (0.6) | (0.7) | (−0.6 to +0.2) | | | | |
| Hct | 41 | 40.9 | 0.0 | −0.1% | NS | 36-48% | |
| | (1.5) | (1.0) | (−1.3 to +1.3) | | | | |
| MCV | 84.55 | 84.49 | −0.05 | −0.1% | NS | 79-98 | fL |
| | (0.6) | (0.7) | (−0.8 to +1.4) | | | | |
| MCH | 28.2 | 28 | −0.54 | −1.9% | <0.001 | 26-32 | pg |
| | (0.9) | (0.9) | (−1.0 to +0.2) | | | | |
| MCHC | 33.6 | 33 | −0.5 | −1.8% | 0.004 | 32-36 | g/dL |
| | (0.5) | (0.5) | (−0.7 to −0.3) | | | | |
| Platelets | 266 | 268 | +1.93 | 0.70% | NS | 140-440 × | |
| | (64.9) | (49.1) | (−25 to +27) | | | 10³/μL | |
| RCDW | 12.6 | 13.2 | +0.5 | 4.40% | 0.0015 | 11.6-15.5% | |
| | (1.0) | (0.8) | (+0.1 to +0.9) | | | | |
| Lymphocytes | 27 | 27 | −0.13 | −0.5% | NS | 20-45% | |
| | (6.7) | (8.1) | (−4.2 to +3.0) | | | | |
| Monocytes | 7 | 7 | 0.0 | 0% | NS | 2-6% | |
| | (0.6) | (0.9) | (−0.4 to +0.4) | | | | |
| Eosinophils | 1 | 2 | +0.3 | 27% | NS | 1-5% | |
| | (0.4) | (0.5) | (0.1 to 0.6) | | | | |
| 25(OH)D | 21.8 | 27.5 | +5.7 | +40.8% | 0.0016 | deficiency: | |
| | (9.9) | (9.3) | (2.4 to 8.0) | | | <10 ng/mL | |
| | | | | | | insufficiency: | |
| | | | | | | 11-20 ng/mL | |
| Venous blood | | | | | | | |
| gas | | | | | | | |
| pH | 7.4 | 7.4 | 0.0 | 0% | NS | 7.32- | |
| | (0.0) | (0.0) | (−0.007 to 0.012) | | | 7.43 | |
| pCO₂ | 51 | 50 | −0.33 | −0.6% | NS | 40-52 | mmHg |
| | (3.8) | (5.0) | (−3.1 to 2.3) | | | | |
| pO₂ | 21.7 | 19.5 | −2.27 | −10.5% | NS | 25-40 | mmHg |
| | (5.8) | (7.7) | (−5.3 to 1.1) | | | | |

SD: standard deviation;
CI: confidence interval;
NS: non-significant;
AST: aspartate aminotransferase;
ALT: alanine aminotransferase;
ALP: alkaline phosphatase;
WBC: while blood cells;
RBC: red blood cells;
HGB: hemoglobin;
Hct: hematocrit;
MCV: mean corpuscular volume;
MCH: mean corpuscular hemoglobin;
MCHC: mean corpuscular hemoglobin concentration;
RCDW: red cell distribution width;
25(OH)D: 25-hydroxyvitamin D

Statistically significant differences between baseline and end of supplementation were found in 7 of the 31 parameters measured. In particular, ALP increased by a mean of 14.2% (p-value<0.0001), but the increased values remained within normal reference ranges for all participants except one whose baseline level was already elevated. Albumin showed a modest decrease by 2.8% (p-value=0.019). Serum potassium levels were reduced by a mean of 4.5% (p-value=0.023), with all decreased values remaining within normal limits. Regarding hematological indices, RSW increased by 4.4% (p-value=0.0015), while MCH and MCHC slightly decreased (−1.9% and −1.8%, respectively; p-value<0.005), all within the normal reference ranges and without any clinical significance. The mean serum 25(OH)D levels increased by 40.8% after the 30-day supplementation (p-value=0.0016), resulting in 14 out of 15 participants with suboptimal vitamin D levels achieving sufficient vitamin D levels by the end of the supplementation period. No other laboratory parameters demonstrated statistically significant changes between Day 0 and Day 30, and no clinically relevant laboratory abnormalities were detected.

### Discussion

To the best of our knowledge, this is the first study of safety and tolerability of a sustained-release ammonium chloride formulation in humans. Our findings indicate that a sustained-release dietary supplement formulation (DIVIRNAM®) administered three times daily for 30 consecutive days was well tolerated and safe in healthy adult individuals.

In our study group, almost three out of four participants developed mild reactions which were self-limited and mainly concerned gastrointestinal symptoms. In particular, all recorded adverse events did not interrupt daily activities and did not require medical attention or discontinuation of the supplement. Overall, no serious adverse events occurred. Indeed, the excellent adherence rate (96.1%) and the high rates of willingness to repeat a 10-day supplementation (93.3%), both underline the very good safety and tolerability profile of DIVIRNAM® at a 50% higher dose than the standard dose and prolonged intake.

Another finding of the current study was the very good laboratory safety profile of the sustained-release ammonium chloride formulation administered at a dose higher than the recommended daily dose and for 30 consecutive days. All observed changes in laboratory parameters were within the normal reference ranges and without any clinical significance. As expected, the mean serum 25(OH)D levels increased markedly after the 30-day supplementation, resulting in 14 out of 15 participants with preexisting suboptimal vitamin D levels achieving sufficient vitamin D levels by the end of the study. Studies indicate that vitamin D ameliorates antiviral immune responses and improves host prognosis, particularly early in the course of a viral infection. This action is achieved through induction of antimicrobial peptides/autophagy and modulation of the reactive hyperinflammatory phase of the host. Vitamin D is expected to provide additional antiviral activity to that provided by ammonium chloride per se. In our study, two individuals noted amelioration of preexisting chronic joint pain, while a third participant reported increased energy levels. These findings can be attributed to the immunomodulatory action of ammonium chloride and deserve further investigation.

The main strength of the present study is its prospective design. One limitation is that all mild events were self-reported, however this is standard practice in safety and tolerability studies. Other limitations are the small number of participants and the fact that individuals with comorbidities were not included in the study.

In conclusion, the present study indicates that a sustained-release ammonium chloride formulation (DIVIRNAM®) administered at a dose of three tablets per day for 30 consecutive days was safe and well tolerated in healthy adult individuals. These findings underscore the need to investigate the effectiveness of DIVIRNAM® in patients with RNA viral infections using higher doses and more prolonged schedules compared to the standard administration.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A formulation for militating against a clinical manifestation of infection by a virus in a subject, the formulation comprising:
a lysosomotropic agent including ammonium chloride in an amount from about 10 mg to about 2,000 mg;
wherein the formulation is configured in a unit dosage form and includes an enteric coating configured for oral administration that provides a sustained release of the ammonium chloride.

2. The formulation according to claim 1, wherein the formulation has a dissolution profile from 4 hours to 24 hours of full release of ammonium chloride.

3. The formulation according to claim 2, wherein the dissolution profile is determined by U.S. Pharmacopeial Convention Monograph USP <711>.

4. The formulation according to claim 1, further comprising a secosteroid.

5. The formulation according to claim 4, wherein the secosteroid includes vitamin D.

6. The formulation according to claim 1, wherein the formulation includes:
the ammonium chloride at 250-500 mg;
vitamin D; and
an excipient including a member selected from a group consisting of steric acid, acetyl alcohol, stearyl alcohol, polyvinylpyrrolidone, magnesium stearate, and combinations thereof.

7. The formulation according to claim 6, wherein the formulation includes:
the ammonium chloride at 250-1,500 mg;
the vitamin D at 1,000-4,000 IU; and
the excipient including steric acid, acetyl alcohol, stearyl alcohol, polyvinylpyrrolidone, and magnesium stearate.

8. The formulation according to claim 1, wherein the lysosomotropic agent is in an amount effective to reduce intracellular viral load at 24 hours post-infection.

9. The formulation according to claim 8, wherein the lysosomotropic agent is in an amount effective to reduce intracellular viral load from 24 hours post-infection up to 72 hours post-infection.

10. The formulation according to claim 9, wherein the lysosomotropic agent is in an amount effective to increase a threshold cycle for detecting intracellular viral load by RT-PCR at 72 hours post-infection by at least 25% for SARS-CoV-2.

11. The formulation according to claim 9, wherein the lysosomotropic agent is in an amount effective to increase a threshold cycle for detecting intracellular viral load by RT-PCR at 72 hours post-infection by at least 50% to 100% for influenza-A.

12. The formulation according to claim 1, wherein:
the formulation has a dissolution profile from 4 hours to 12 hours of full release of ammonium chloride and the dissolution profile is determined by U.S. Pharmacopeial Convention Monograph USP <711>;
the formulation includes the ammonium chloride at 250-1,500 mg, vitamin D, and an excipient including a member selected from a group consisting of steric acid, acetyl alcohol, stearyl alcohol, polyvinylpyrrolidone, magnesium stearate, and combinations thereof; and
the lysosomotropic agent is in an amount effective to reduce intracellular viral load at 24 hours post-infection.

13. A method of militating against a clinical manifestation of infection by a virus in a subject, the method comprising:
administering a formulation to the subject, the formulation including a lysosomotropic agent having ammonium chloride in an amount from about 10 mg to about 2,000 mg, the formulation configured in a unit dosage form, and the formulation having an enteric coating configured for oral administration that provides a sustained release of the ammonium chloride;
monitoring the subject for at least one of alkalosis and acidosis, wherein the monitoring includes measuring a blood pH of the subject;
selectively adjusting the administering of the formulation to the subject based on the monitoring so that the subject is not exhibiting alkalosis and is not exhibiting acidosis, wherein the selectively adjusting includes adjusting the administering of the formulation to the subject until the blood pH of the subject is between about 7.35 and about 7.45; and
continuing the administering of the formulation over a period of at least 24 hours to reduce intracellular viral load at 24 hours post-infection.

14. The method according to claim 13, wherein continuing the administering of the formulation includes continuing administration of the formulation over a period of 72 hours to reduce intracellular viral load at 72 hours post-infection.

15. The method according to claim 13, wherein the formulation has a dissolution profile from 4 hours to 24 hours of full release of ammonium chloride.

16. The method according to claim 13, wherein the formulation includes:
the ammonium chloride at 250-500 mg;
vitamin D; and
an excipient including a member selected from a group consisting of steric acid, acetyl alcohol, stearyl alcohol, polyvinylpyrrolidone, magnesium stearate, and combinations thereof.

17. The method according to claim 13, wherein the virus includes SARS-CoV-2 and the lysosomotropic agent is in an amount effective to increase a threshold cycle for detecting intracellular viral load by RT-PCR at 72 hours post-infection by at least 10% to 25% for SARS-CoV-2.

18. The method according to claim 13, wherein the virus includes influenza-A or influenza-B and the lysosomotropic agent is in an amount effective to increase a threshold cycle for detecting intracellular viral load by RT-PCR at 72 hours post-infection by at least 50% to 100% for influenza-A or influenza-B.

19. The method according to claim 13, further comprising continuing the administration of the formulation to the subject until the virus cannot be detected in the subject.

20. The method according to claim 13, further comprising continuing the administration of the formulation to the subject until an antibody to the virus is detected in the subject.
